(19) United States
(12) Patent Application Publication
(10) Pub. No.: US 2021/0216925 A1
(43) Pub. Date: Jul. 15, 2021

(54) GENERATING A PROCESS MODEL

(71) Applicant: KONINKLIJKE PHILIPS N.V., EINDHOVEN (NL)

(72) Inventors: Prabhakar Dixit, Eindhoven (NL); Johannes Buurman, Eindhoven (NL)

(21) Appl. No.: 16/477,896

(22) PCT Filed: Jan. 17, 2018
(86) PCT No.: PCT/IB2018/050275

§ 371 Date: Jul. 14, 2019

Related U.S. Application Data
(60) Provisional application No. 62/585,714, filed on Nov. 14, 2017.
(60) Provisional application No. 62/568,947, filed on Oct. 6, 2017.
(60) Provisional application No. 62/450,140, filed on Jan. 25, 2017.

(51) Int. Cl. G06Q 10/06; G16H 40/20; G16H 50/70

(52) U.S. Cl. CPC G06Q 10/0633; G16H 50/70; G16H 40/20

(57) ABSTRACT

The invention discloses a computer-implemented method of generating a process model. The method comprises obtaining, from a database, details of a first plurality of activities to be included in the process model; receiving, via a user input, details of a second plurality of activities to be included in the process model; generating the process model based on the details of the first and second plurality of activities; and displaying the generated process model to a user.

### CROSS-REFERENCE TO RELATED APPLICATIONS

The present application claims priority to and benefit of both U.S. Provisional Application No. 62/450,140, filed Jan. 25, 2017, and U.S. Provisional Application No. 62/568,947, filed Oct. 6, 2017, their entirety of which is hereby incorporated by reference herein.

### FIELD OF THE INVENTION

The invention relates to process modelling and, in particular to a method and apparatus for generating a process model.

### BACKGROUND OF THE INVENTION

Process modelling is a technique used to describe or predict how a process should be performed, or what the process will look like. A process model may be created or generated using existing data, such as data relating to processes that have been performed in the past. Generating a process model in this way is referred to as process discovery, or process model discovery. Process discovery is a data mining technique which mines data (e.g. an event log) in an information system and constructs a process model using data acquired from the information system.

While process modelling may be used in a wide range of technical sectors, one area in which process modelling may find particular benefits is in the medical field. A clinical care pathway (also referred to simply as a clinical pathway or care pathway) is a plan describing a process to be performed in respect of a patient in a clinical environment, such as in a hospital. A clinical care pathway may set out a standardized, or recommended set of actions or processes to be performed in respect of a patient suffering from a particular medical condition. For example, a clinical care pathway for a patient suffering from a particular type of cancer may define a set of actions including sending the patient for a scan, taking a blood sample, prescribing a particular medication, administering the medication at a particular time, and so on. Such a standard clinical care pathway may have been created by a group of experts, based on past experience.

However, such standardized clinical care pathways are not suitable for all patients. A particular patient may be suffering from multiple medical conditions (referred to a comorbidity), so a standardized clinical care pathway for one medical condition may not be compatible with a clinical care pathway for another medical condition. In the broader sense, one standard process may not be compatible with another standard process to be performed concurrently.

Another way of generating a process model, which may, for example, be used to construct a clinical care pathway, is to obtain input from multiple participants who perform some role in the process. For example, a process model may be constructed by querying medical professionals who are likely to be involved in the treatment of a patient suffering from a particular type of cancer. Each medical professional may provide a list of actions that need to be included in a clinical care pathway. The multiple lists may then be compiled into a single clinical care pathway, or process model. This method of process modelling may have a disadvantage in that person obtaining the information from the medical professionals may miss some critical information which plays a key role in the overall process. Alternatively, the medical professionals may anticipate some prior knowledge of the person obtaining the information, and circumvent some important details. The inappropriate information collected could lead to cases where a clinical care pathway is modelled, but which is an inaccurate reflection of the process that should take place and, hence, may result in an ineffective care pathway design.

Therefore, there is a need for a method for generating a process model in which a process model can be constructed in a more flexible way, and which helps to address some of the above issues.

### SUMMARY OF THE INVENTION

It would be beneficial to be able generate a more flexible process model, which includes additional inputs. The inventors have discovered that a more flexible process modelling technique may be achieved if some activities to be performed are obtained from a database (e.g. a database containing previously-generated process models) and other activities are input by a user, such as a professional who is skilled in the relevant field. A process model generated in this way may be tailored in a particular way (e.g. tailored to a particular individual) for which a standardized existing process model is not appropriate.

According to a first aspect, the invention provides a computer-implemented method of generating a process model. The method comprises obtaining, from a database, details of a first plurality of activities to be included in the process model; receiving, via a user input, details of a second plurality of activities to be included in the process model; generating the process model based on the details of the first and second plurality of activities; and displaying the generated process model to a user. In this way, an interactive process model generation technique may be achieved. This allows a user to tailor a process to a particular situation in which a standard process may not be suitable.

In some embodiments, the method may comprise obtaining, from a database, details of where in the process model activities are to be included. Similarly, the method may comprise receiving, via a user input, details of where in the process model activities are to be included.

The database may, in some embodiments, comprise an event log, the event log including information regarding activities that have been performed in relation to at least one previously-performed process.

The information may comprise at least one of: information regarding the nature of an activity that has been performed; information regarding when an activity has been performed in the previously-performed process; and information regarding a subject in respect of whom an activity has been performed.

In some embodiments, the method may further comprise presenting to a user a third plurality of activities, the third plurality of activities including options of activities to be included in the process model.

The method may further comprise ordering the third plurality of activities according to one or more metrics. In some embodiments, the one or more metrics may be selected from a group comprising: a fitness metric, defining, for each activity in the third plurality of activities, a goodness of fit for the activity in the process model; a precision metric, defining, for each activity in the third plurality of activities, a preciseness of the process model, if the activity is included in the process model, as compared to a previously-performed process; a complexity metric, defining, for each activity in the third plurality of activities, a degree of complexity of the process model if the activity is included in the process model; and a knowledge-based metric, defining, for each activity in the third plurality of activities, at least one requirement that must be met.

The method may, in some embodiments, comprise modifying the displayed process model based on a user selection of an activity from the third plurality of activities to be included in the process model.

In some embodiments, the method may comprise presenting to a user a recommended activity to be included in the process model. For example, the recommended activity may be selected from a plurality of activities, the selection of the recommended activity being made based on at least one of: an alphabetical policy, whereby the recommended activity comprises the next activity of the plurality of activities when ordered alphabetically; and a heuristics policy, whereby the recommended activity comprises an activity which occurs most commonly in previously-performed processes.

The method may further comprise modifying the displayed process model to include the recommended activity.

The process model may, in some embodiments, comprise a model representing a clinical care pathway.

In some embodiments, each activity in the plurality of activities may comprise one or more steps to be performed.

According to a second aspect, the invention provides a computer program product comprising a non-transitory computer readable medium, the computer readable medium having computer readable code embodied therein, the computer readable code being configured such that, on execution by a suitable computer or processor, the computer or processor is caused to perform the methods described herein.

According to a third aspect, the invention provides an apparatus comprising a display and a processor. The processor is configured to obtain, from a database, details of a first plurality of activities to be included in the process model; receive, via a user input, details of a second plurality of activities to be included in the process model; generate the process model based on the details of the first and second plurality of activities; and display the generated process model to a user.

These and other aspects of the invention will be apparent from and elucidated with reference to the embodiments described hereinafter.

### BRIEF DESCRIPTION OF THE DRAWINGS

For a better understanding of the invention, and to show more clearly how it may be carried into effect, reference will now be made, by way of example only, to the accompanying drawings, in which:

FIG. 1 is a schematic illustration of an example of an arrangement for generating a process model;

FIG. 2 is a flowchart of an example of a method of generating a process model;

FIG. 3 is a flowchart of a further example of a method of generating a process model;

FIG. 4 is a flowchart of a further example of a method of generating a process model;

FIG. 5 is a representation of an example of a user interface;

FIG. 6 is a flowchart of a further example of a method of generating a process model;

FIG. 7 is a representation of an example of part of the user interface of FIG. 5;

FIG. 8 is a flowchart of a further example of a method of generating a process model;

FIG. 9 is a flowchart of a further example of a method of generating a process model;

FIG. 10 is a schematic illustration of an example of an apparatus for generating a process model;

FIG. 11 is a schematic illustration of a machine-readable medium and a processor;

FIGS. 12 to 32 are Figures referred to in Annex A; and

FIGS. 33 to 40 are Figures referred to in Annex B.

### DETAILED DESCRIPTION OF EMBODIMENTS

The invention provides an improved mechanism by which a process model may be generated. A process model is a model that describes how a process is to be performed. A process model may include steps or activities that are to be performed as part of the process, and may describe the way in which the various activities relate to one another. For example, the process model may describe an order in which particular steps are to be performed and/or any consequences that result from a particular activity being performed, or not being performed.

While embodiments of the invention are described in terms of medical process models, such as clinical care pathways, the disclosed methods, apparatus and systems are applicable to as wide range of other fields. In general, the invention is applicable to any field in which a process to be performed is to be modelled. For example, the invention may find application in the fields of manufacturing, project planning, operational research, data monitoring and auditing, production systems and the like.

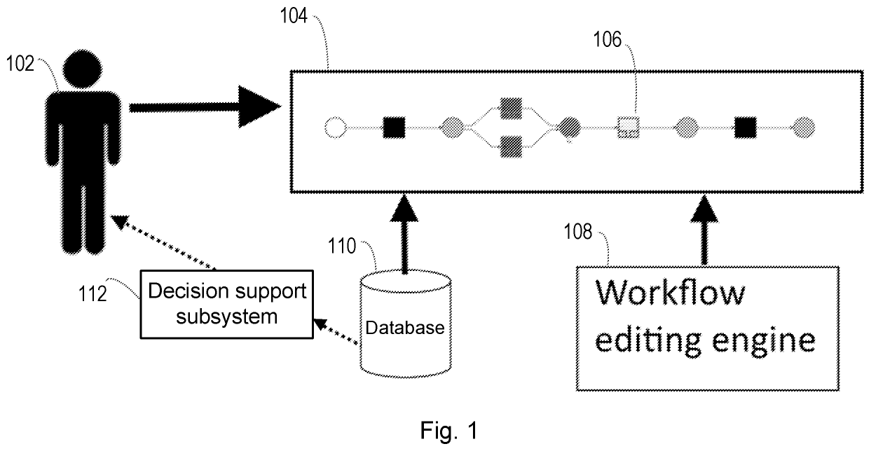

FIG. 1 shows an example of the manner in which a user may interact with a system to generate a process model in the context of the invention. FIG. 1 shows a user 102 who is able to interact with a process model discovery system 104 in order to generate a process model 106. The process model discovery system 104 may comprise a computer program or application which can be executed using suitable processing circuitry (e.g. processor) of a computing device, such as a desktop, laptop or tablet computer, or on a mobile device such as a smartphone. The user 102 may interact with the process model discovery system 102 (and the computing device on which the system is executed) via a user interface, which may include a keyboard, a mouse, a touchpad, a touchscreen or the like. The process model discovery system 104 may be referred to as a process model editor, as this may be the mechanism by which the user 102 is able to create and edit the process model 106.

In addition to receiving inputs from the user 102, the process model discovery system 104 may receive inputs from a workflow editing engine 108 and/or a database 110. The workflow editing engine 108 and the database 110 may form part of the same computing device on which the process model discovery system 104 operates, or may be remote from the process model discovery system, and connected to, accessible by and/or in communication with the process model discovery system via wired or wireless connection.

The workflow editing engine 108 ensures that any process model that is generated in the process model discovery system 104 is semantically sensible, and capable of functioning. For example, the workflow editing engine 108 may prevent a process model from being generated which does not have a defined end point, or which could get stuck in a loop (e.g. if an output from an activity is modelled to be fed back into the same activity as an input, without any other outputs).

The database 110 may contain data or information which can be incorporated into the process model 106. For example, the database 110 may contain previous-generated process models and/or at least parts of other process models, which may be used as the basis of the process model 106 being generated in the system 104, or which may otherwise be incorporated into the process model 106. In some embodiments, for example those in which the process model 106 comprises a clinical care pathway, the database 110 may comprise an event log. An event log is a record of activities that have taken place in respect of an entity (also referred to as a case). For example, an event log may record activities that have taken place in respect of a particular medical patient, or a particular medical facility (e.g. a hospital), or a particular piece of medical equipment (e.g. an Mill scanning system). Such an event log may include a time stamp for each activity so that it is possible to determine when each activity was performed, and the order in which each of the activities took place. The database 110, or event log, may further include additional information relating to the activities include therein.

Information from the database 110 may also be accessible by the user 102, for example via a decision support subsystem 112. The decision support subsystem 112 may provide information to the user 102, or maybe accessible by the user, in order to assist the user in creating and/or editing the process model in the process model discovery system 104. For example, the user may identify a particular activity which has been performed in respect of a majority of patients suffering from a particular medical condition, but which is not included in a standard clinical care pathway for that medical condition. The user 102 may, therefore, decide to include that activity in the process model 106.

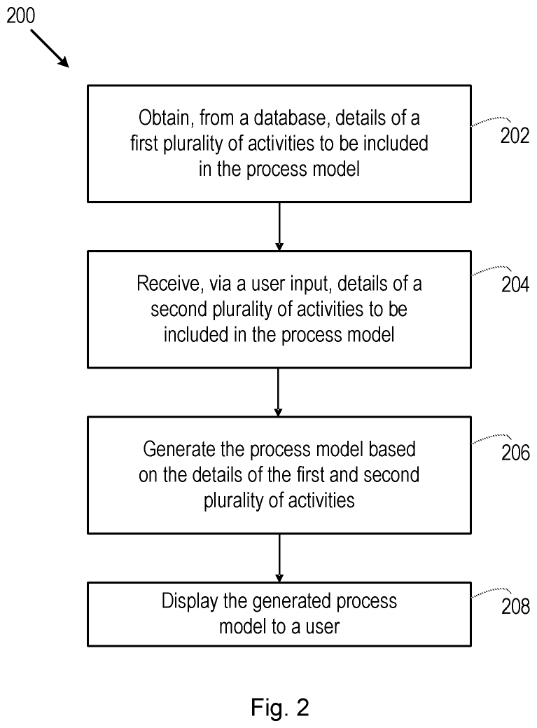

FIG. 2 is a flowchart of an example of a computer-implemented method 200 for generating a process model, such as the process model 106. The method 200 comprises, at step 202, obtaining, from a database (e.g. the database 110), details of a first plurality of activities to be included in the process model. The first plurality of activities may be in the form of a previously-generated process model or a previously-performed process. At step 204, the method comprises receiving, via a user input, details of a second plurality of activities to be included in the process model. The second plurality of activities may be activities of which a user (e.g. the user 102) is aware, for example from his or her personal or professional knowledge, or from information acquired from the database. The method comprises, at step 206, generating the process model based on the details of the first and second plurality of activities. In some embodiments, the generation of the process model may be performed by, or with assistance from, the workflow editing engine 108 discussed above. In a simple example, generating the process model may comprise combining or assembling the first and second pluralities of activities into a single process model. In other examples, the generation of the process model may be more complex, and may involve complex paths between activities depending on numerous parameters and/or factors. The way in which the process model is generated may depend on parameters defined by the user 102, on parameters defined in the process model discovery system 104 itself, and/or on rules, requirements and/or preferences programed into the system, or provided, for example, by the workflow editing engine 108. The generation of a process model, and the functionality provided by the workflow editing engine 108 are discussed below.

At step 208, the method 200 comprises displaying the generated process model to a user. The generated process model may be displayed using a suitable display device, such as a display screen. The display device may form part of the process model discovery system 104, or may be remote from, but associated with and/or connected to, the system.

Using the method 200 described above, the user 102 is able to generate a personalized, or bespoke, process model which defines a process for a particular situation, and based on a particular set of parameters. Compared to other methods, in which a process model may be created using input solely from individuals (e.g. medical professionals), the method 200 provides a more versatile mechanism for creating a process model, and allows for the creation of a more effective process model which is based not only on experiences, opinions and views of other users, but also on data relating to existing, tried and tested processes. A generated process model can be run (i.e. executed) using data from an existing or previously-performed process (e.g. data from the event log), in order to determine the location of any bottlenecks in the generated process model. In a medical environment, the generated process model may be used to identify any deviations in the process defined by the process model. For example, the process model may be used to determine whether any patients for which the process is not followed. A further advantage of the disclosed method is that a “correct” or optimum process model can be generated, which enables accurate analysis of data. As such, processes may be further developed and optimized over time.

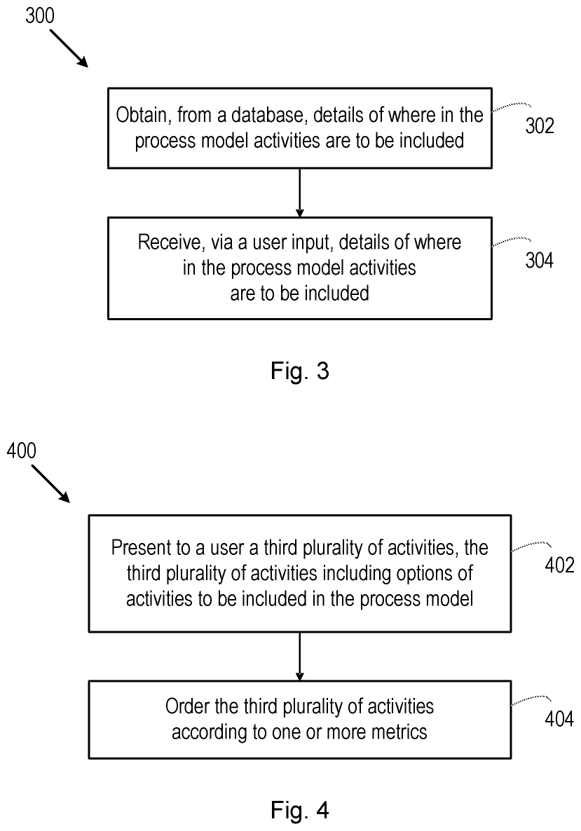

FIG. 3 is a flowchart of a further example of a computer-implemented method 300 for generating a process model. The method 300 may comprise steps of the method 200. At step 302, the method 300 may further comprise obtaining, from a database, details of where in the process model activities are to be included. The database may comprise the same database from which the details of the first plurality of activities is obtained (i.e. in step 202). In some embodiments, the database may comprise the database 110. The details obtained at step 302 may define a position at which a particular activity is to be included in the process model 106. The position may be defined in absolute or relative terms. For example, the position may be defined relative to other activities included in the process model 106.

At step 304 the method 300 may further comprise receiving, via a user input, details of where in the process model activities are to be included. Thus, information similar to that received at step 302 may be received via a user input rather than from the database. In some embodiments, the information may be provided by a user (e.g. the user 102) via a user interface associated with the process model discovery system 104. For example, the user 102 may enter the intended position of a particular activity on a representation of the process model, using an input device, such as a mouse.

According to some embodiments, the database may comprise an event log. As discussed above, the event log may include information regarding activities that have been performed in relation to at least one previously-performed process. In some embodiments, the event log may include information regarding activities that have been included in at least one previously-generated process model. When the claimed method is applied to the medical field, the event log may include details of medical activities (e.g. medical procedures, medical tests, medical imaging scans, and the like) that have been performed in respect of a patient suffering from one or more medical conditions. Some of the information (e.g. an activity which appears to have been particularly effective in treating a medical condition) may be included in the process model 106 to be generated.

In some embodiments, the information included in the event log may comprise at least one of: information regarding the nature of an activity that has been performed; information regarding when an activity has been performed in the previously-performed process; and information regarding a subject in respect of whom an activity has been performed. The event log may include information falling within any one or two of these categories, or information from all three categories.

Information regarding the nature of an activity that has been performed may include details of what the previously-performed activity is. Information regarding when an activity has been performed in the previously-performed process may include, in absolute or relative terms, details of the position in the previously-performed process the activity took place. This may provide an insight as to when the activity is to be performed to achieve the most effective result from the process. Information regarding a subject in respect of whom an activity has been performed may include data about an individual or group of individuals to whom the performed activity relates. For example, the information may include medical data relating to a person to whom an activity (e.g. medical procedure) has been performed.

FIG. 4 is a flowchart of a further example of a computer-implemented method 400 for generating a process model. The method 400 may comprise steps of the methods 200, 300. The method 400 may further comprise, at step 402, presenting to a user a third plurality of activities, the third plurality of activities including options of activities to be included in the process model. The third plurality of activities may additional activities which are not already included in the process model 106, one or more of which may be additionally included. The activities included in the third plurality of activities may be provided by obtained from the database (e.g. the database 110), and/or received via the user input (e.g. from the user 102). A user may select (e.g. using a mouse) one or more of the third plurality of activities presented to them, to cause the selected activity or activities to be added into the process model 106.

At step 404, the method 400 may further comprise ordering the third plurality of activities according to one or more metrics. In other words, to enable the user to make a more informed decision regarding which, if any, activity or activities to incorporate into the process model 106 being generated, the method 400 involves ordering (or re-ordering) or ranking the activities. The order (or rank) in which the activities are presented may depend on one or more factors or metrics. In some embodiments, for each activity in the third plurality of activities, the method may involve determining a selection of positions at which each activity may be added into the process model. The selection of possible positions may be presented to the user, and may be ordered or ranked according to one or metrics.

The metrics used to order the third plurality of activities or the possible positions of the activities may depend on numerous factors, such as the intended application of the process model, the experience level of the user involved in generating the process model, and the nature of the information available. However, in some embodiments, the one or more metrics may be selected from a group comprising: a fitness metric, a precision metric, a complexity metric, and a knowledge-based metric. The fitness metric, or goodness of fit metric defines, for each activity in the third plurality of activities, a goodness of fit for the activity in the process model. In other words, the fitness metric provides a measure of how well data from the database (e.g. an event log) fits the process model. The precision metric defines, for each activity in the third plurality of activities, a preciseness of the process model, if the activity is included in the process model, as compared to a previously-performed process. For example, an activity may be rated lower according to the precision metric if it would cause the process model to require additional behavior (e.g. extra tasks or activities beyond the activity to be added). The complexity metric defines, for each activity in the third plurality of activities, a degree of complexity of the process model if the activity is included in the process model. Using this metric, an activity which could be introduced into the process metric in a relatively more simple manner (e.g. resulting in a less complex process model) is likely to rate higher than an activity whose addition would result in a more complex process model. The knowledge-based metric defines, for each activity in the third plurality of activities, at least one requirement that must be met. Such a requirement may be based on the knowledge of an expert, or on a particular requirement set by the user. For example, a user may require that a particular activity can only be added to the process model if it does not involve the patient moving. Those activities which do not meet the defined requirement (e.g. which do require the patient to move) would rate relatively lower according to this metric.

The metrics may be scored between 0 and 1. In some embodiments, other metrics, for example metrics selected by the user based on the circumstances of the situation, may be introduced and used to order the third plurality of activities. One or more than one metric may be used, including any combination of those metrics discussed above.

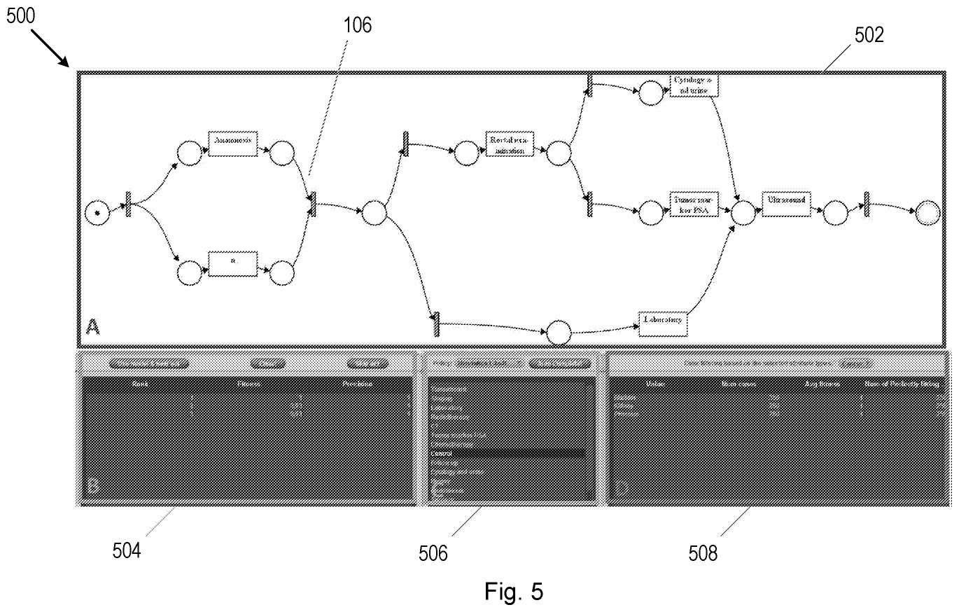

FIG. 5 shows an example of a graphical user interface 500 for use in generating the process model, for example, using the methods disclosed herein. The user interface 500 may, for example, be displayed to a user 102 on the display screen of a computing device on which the process model discovery system 104 operates.

The user interface 500 includes a process model view window or panel 502, a ranking window or panel 504, an activity selection window or panel 506 and an analysis window or panel 508. The process model view window 502 may display the process model 106 as it is being created. The process model 106 shown in the window 502 includes a start node 510, an end node 512 and various activity nodes 514a to 514g between the start and end nodes, each representing an activity to be performed as part of the process being modelled. The nodes may be linked, shown in FIG. 5 with arrows representing the order in which the activities are to be performed. Within the process model 106, a particular vertical rectangular node 516 represents a split in the process path into two separate paths, both of which should be followed, but in any order. A particular circular node 518 represents a split in the process path into two separate paths, either of which can be followed. Other rectangular and circular nodes included in the process model 106 may be assigned other meanings.

When steps 202 and 204 of the method 200 have been completed (i.e. details of the first and second plurality of activities have been acquired), the process model 106 may be generated (step 206) and displayed (step 208) to the user 102 in the process model view window 502. In the embodiment shown in FIG. 5, a third plurality of activities may be displayed in the activity selection window 506. The third plurality of activities may, in some embodiments, comprise activities suggested or recommended for inclusion in the process model 106 by the system. In order to add an additional activity into the process model 106, the user may, for example, scroll through the third plurality of activities and select an activity, for example using a selection tool in the graphical user interface. Once an activity has been selected, a list of possible or recommended positions may be displayed in the ranking window 504. The ranking window may present various options for positions in the process model at which the selected activity may be added, along with a score for each metric being used in the ranking. In the example shown in FIG. 5, three possible options are shown in the ranking window, with the highest ranked option shown at the top of the list. The highest ranked option has fitness and precision scores of 1, meaning that the highest ranked option would provide the most suitable position for inserting the selected activity into the process model.

The analysis window 508 displays a measure of the relevance of the process model to different groups of people (i.e. different cases). The analysis window 508 allows a user to select a category such as disease type, age, gender, and the like on which to perform analysis. Adding activities into the process model 106 will change the process model and, therefore, will change the relevance of the process model to various cases in the selected categories. This allows a user to see easily how relevant the currently-displayed process model is to the particular group of cases shown in the analysis window 508.

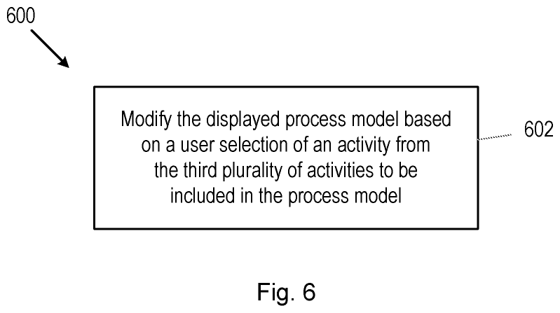

FIG. 6 is a flowchart of a further example of a computer-implemented method 600 of generating a process model. The method 600 may include steps of other methods disclosed herein. The method 600 may further comprise, at step 602, modifying the displayed process model based on a user selection of an activity from the third plurality of activities to be included in the process model. Thus, if a user selects an activity from the third plurality of activities (e.g. in the selection window 506), then the process model 106 may be modified to show the user how the process model would change with the addition of the selected activity. In some embodiments, the process model 106 may be further modified or updated in response to the user selecting an alternative position for the activity to be added into the process model, for example by making a selection of one of the ranked positions shown in the ranking window 504.

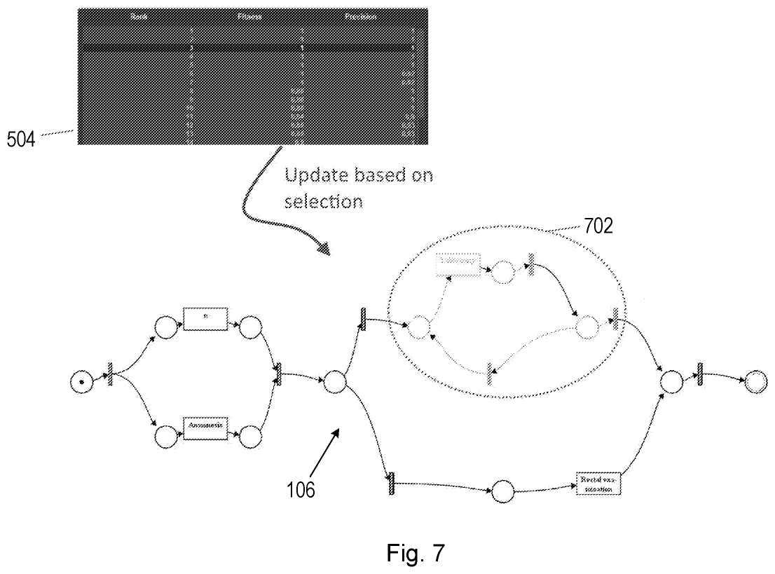

An example of how the user interface 500 may display the modified process model 106 is shown in FIG. 7. FIG. 7 shows a portion of a ranking window 504 which, in this example, includes at least fourteen options for possible positions in the process model at which an activity could be added. Selecting one of the options (e.g. the option ranked at 3 in the example shown) causes the selected activity to be added into the process model 106 at the chosen position. In FIG. 7, dashed ring 702 indicates the part of the process model 106 that is changed as a result of adding the selected activity at the selected position (i.e. the position ranked at 3). In this way, a user 102 is able to see how the process model 106 is likely to change (and what other consequences will occur) depending on the activity and/or position selected.

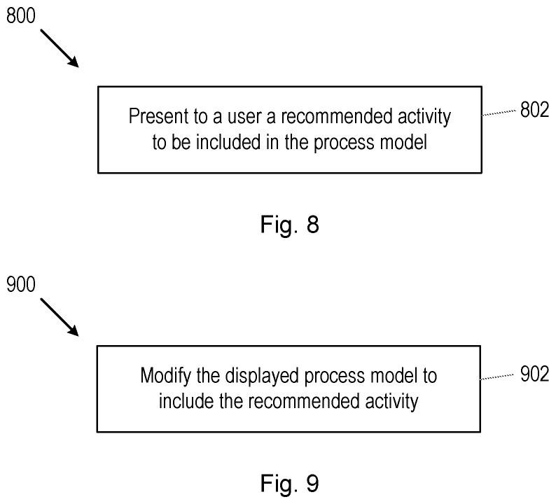

In some embodiments, one or more recommendations of activities to be included in the process model may be made and suggested to the user 102. FIG. 8 is a flowchart of a further example of a method 800 of generating a process model. The method 800 may include steps of other methods disclosed herein. The method 800 may comprise, at step 802, presenting to a user a recommended activity to be included in the process model. The recommended activity may, for example, be based on an activity included in the database 106 or in an event log.

In some embodiments, the recommended activity may be selected from a plurality of activities, the selection of the recommended activity being made based on at least one of: an alphabetical policy, whereby the recommended activity comprises the next activity of the plurality of activities when ordered alphabetically; and a heuristics policy, whereby the recommended activity comprises an activity which occurs most commonly in previously-performed processes. For example, using a heuristics policy, an activity may be recommended which is most likely to be required next in the process, based on other previously-performed processes, or based on previously-generated process models stored in the database.

If a user accepts a recommended activity, then the recommended activity may be incorporated into the process model 106. FIG. 9 is a flowchart of a further example of a method 900 of generating a process model. The method 900 may include steps of other methods disclosed herein. The method 900 may comprise, at step 902, modifying the displayed process model to include the recommended activity. The modification of the process model may be presented in a manner similar to that shown in FIG. 7, for example, with the modified portion highlighted or indicated to the user 102.

The methods described herein may be implemented in a number of ways by a user to interactively create a process model. As the process model is created, the user is able to tell in real time the effect of adding a new activity at a particular position in the process model.

The user 102 may implement the method (e.g. using the apparatus 1100) by having the system recommend activities to be included in the process model 106. For example, based on a patient profile (which may identify a medical condition from which the patient is suffering) a first activity may be suggested. If accepted by the user 102, that activity will be added to the process model and, based on that activity, the patient profile, and information on previously-generated process models (e.g. in an event log), a second activity may be suggested, and so on. In such an example, a user 102 may have relatively little input.

In another example, some of the activities to be included in the process model may be recommended by the system, while others may be selected by the user 102. The user 102 may decide where in the process model each activity is to be positioned, and may take advice (e.g. recommendations) of the positions from the system, for example based on information acquired from the database or event log. In such an example, the user 102 may switch between allowing the system to create the process model (known as automated discovery) and providing user input (known as interactive discovery). For example, the user 102 may decide to build the process model to a certain point, based on their knowledge and experience, then allow the system to build the rest of the process model in an automated procedure.

Automated discover of a process model may be achieved using known techniques; for example, known synthesis rules for Petri nets may be used to generate the process model.

As discussed previously, the general disclosed method of generating a process model may be used in the medical field for generating clinical care pathways. Thus, in some embodiments, the process model may comprise a model representing a clinical care pathway. In any embodiment, each activity in the plurality of activities may comprise one or more steps to be performed. When applied to the medical field, the activities may relate to medical tasks to be performed in relation to a patient.

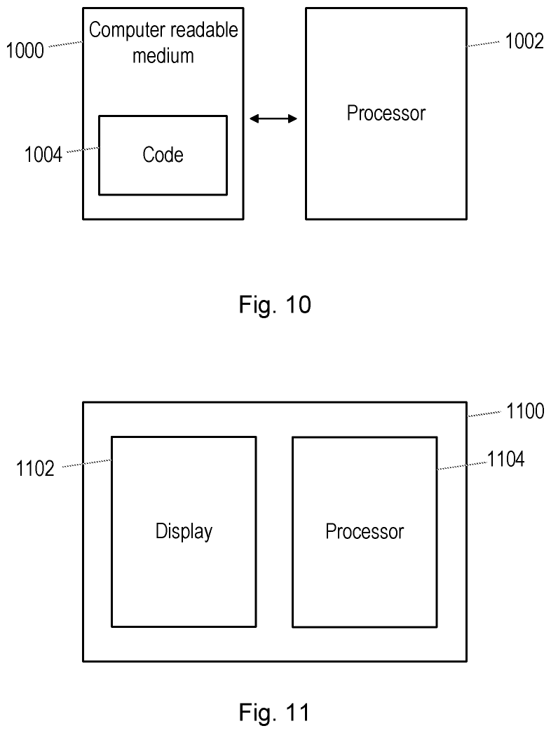

In addition to the methods disclosed herein, an aspect of the invention relates to a computer program product. The methods described herein may be performed by a computer, and may be implemented using computer-executable code. FIG. 10 is a schematic illustration of an example of a computer readable 1000 and a processor 1002. A computer program product comprises a non-transitory computer readable medium 1000, the computer readable medium having computer readable code 1004 embodied therein, the computer readable code being configured such that, on execution by a suitable computer or processor 1002, the computer or processor is caused to perform the methods disclosed herein.

In addition to the computer program product, an aspect of the invention relates to an apparatus. FIG. 11 is a schematic representation of an example of an apparatus 1100. The apparatus 1100 comprises a display 1102 and a processor 1104. The processor 1104 is configured to obtain, from a database, details of a first plurality of activities to be included in the process model; receive, via a user input, details of a second plurality of activities to be included in the process model; generate the process model based on the details of the first and second plurality of activities; and display the generated process model to a user. The apparatus 1100 may, in some embodiments, comprise a computing device.

The processor 1104 can comprise one or more processors, processing units, multi-core processors or modules that are configured or programmed to control the apparatus 1100 in the manner described herein. In particular implementations, the processor 1104 can comprise a plurality of software and/or hardware modules that are each configured to perform, or are for performing, individual or multiple steps of the method described herein.

The term “module”, as used herein is intended to include a hardware component, such as a processor or a component of a processor configured to perform a particular function, or a software component, such as a set of instruction data that has a particular function when executed by a processor.

It will be appreciated that the embodiments of the invention also apply to computer programs, particularly computer programs on or in a carrier, adapted to put the invention into practice. The program may be in the form of a source code, an object code, a code intermediate source and an object code such as in a partially compiled form, or in any other form suitable for use in the implementation of the method according to embodiments of the invention. It will also be appreciated that such a program may have many different architectural designs. For example, a program code implementing the functionality of the method or system according to the invention may be sub-divided into one or more sub-routines. Many different ways of distributing the functionality among these sub-routines will be apparent to the skilled person. The sub-routines may be stored together in one executable file to form a self-contained program. Such an executable file may comprise computer-executable instructions, for example, processor instructions and/or interpreter instructions (e.g. Java interpreter instructions). Alternatively, one or more or all of the sub-routines may be stored in at least one external library file and linked with a main program either statically or dynamically, e.g. at run-time. The main program contains at least one call to at least one of the sub-routines. The sub-routines may also comprise function calls to each other. An embodiment relating to a computer program product comprises computer-executable instructions corresponding to each processing stage of at least one of the methods set forth herein. These instructions may be sub-divided into sub-routines and/or stored in one or more files that may be linked statically or dynamically. Another embodiment relating to a computer program product comprises computer-executable instructions corresponding to each means of at least one of the systems and/or products set forth herein. These instructions may be sub-divided into sub-routines and/or stored in one or more files that may be linked statically or dynamically.

The carrier of a computer program may be any entity or device capable of carrying the program. For example, the carrier may include a data storage, such as a ROM, for example, a CD ROM or a semiconductor ROM, or a magnetic recording medium, for example, a hard disk. Furthermore, the carrier may be a transmissible carrier such as an electric or optical signal, which may be conveyed via electric or optical cable or by radio or other means. When the program is embodied in such a signal, the carrier may be constituted by such a cable or other device or means. Alternatively, the carrier may be an integrated circuit in which the program is embedded, the integrated circuit being adapted to perform, or used in the performance of, the relevant method.

Variations to the disclosed embodiments can be understood and effected by those skilled in the art in practicing the claimed invention, from a study of the drawings, the disclosure and the appended claims. In the claims, the word “comprising” does not exclude other elements or steps, and the indefinite article “a” or “an” does not exclude a plurality. A single processor or other unit may fulfil the functions of several items recited in the claims. The mere fact that certain measures are recited in mutually different dependent claims does not indicate that a combination of these measures cannot be used to advantage. A computer program may be stored/distributed on a suitable medium, such as an optical storage medium or a solid-state medium supplied together with or as part of other hardware, but may also be distributed in other forms, such as via the Internet or other wired or wireless telecommunication systems. Any reference signs in the claims should not be construed as limiting the scope.

The following text describes specific details relevant the invention.

### ANNEX A

### Interactive Process Discovery Using Domain Knowledge and Process Mining

In healthcare setting, there is a growing need for standardizing the care pathways, for improving operational efficiency. Sometimes, such care pathways are explicitly defined by the hospitals, and it is expected that the practitioners adhere to these care pathways. However, more often than not such care pathways aren't very well defined. Owing to the benefits of a standardized care process, more and more institutions are moving towards introducing a standardized care pathway. However, learning the care pathway if far from trivial. Most of the steps performed in such care pathways are recorded by the information systems in the hospital. Process discovery techniques could be used on such event logs, in order to learn the care pathway. However, the data is usually noisy and/or incomplete, and hence process mining techniques cannot be used out of the box. Therefore, in this invention, we propose a way to model the care pathway process, in an interactive way, using the information from event log to guide the modelling process.

### BACKGROUND

This disclosure relates to process model discovery and editing. Modelling the discovery of such clinical care pathways is a challenge. There are products which record most of the steps that constitute to the clinical care pathway.

Traditionally, the modelling of clinical care pathways is done by interviewing the relevant participants which play some role in the process, and putting all their stories together to model an end to end process model. This may have the disadvantage that the interviewer (consultant) may miss some critical information which plays a key role in the overall process. Or, the participants may anticipate some prior knowledge of the interviewer and circumvent some important details. The inappropriate information collected could lead to cases wherein the care pathway modelled by the interviewer to be the incorrect reflection of the actual reality, and hence result in an ineffective care pathway design.

Recently, a lot of emphasis has been put up on discovering process models using the event logs which record the information performed during each step of the clinical care pathway. Such techniques, called process discovery techniques, tend to rely entirely on the event logs to learn the most plausible description of the clinical care pathway by using machine learning and/or statistical analysis. However, the data recorded may contain too little or too much information. Furthermore, the event logs may contain noisy and or incomplete information, which could lead to the discovery of incorrect process models. Moreover, if there are a lot of activities, the discovered process model might become unreadable, and can only be used for analysis purposes and not in reality for modelling a clinical pathway.

### DETAILED DESCRIPTION

It would be ideal to combine the two worlds of modelling care pathways, and automatically discovering them using event logs. In literature, there has been some focus in order to achieve this, by repairing a process model. That is, using a pre-existing hand-drawn care pathway model and combining it with the event log data to repair it to reflect the reality. However, in practical settings, all the disadvantages mentioned above corresponding to the incompleteness/noisy notion of the data still persist. In order to overcome this, we suggest an interactive process discovery/modelling approach, wherein the user can interactively explore and model the care pathway by effectively combining the domain expertise with the event log information.

In order to enable interactive process modelling/discovery, we devise a system containing primarily of three components, as described below:
1. The workflow editing engine: This engine guarantees the generation of semantically sensible process models. Depending on the requirements, these models could be used either as protocols, or could be made executable. These properties determine the usage of workflow editing engine to be used.
2. The main editor: This is the primary interactive modelling editor, which the user interacts with, in order to discover process models. The care pathways modelled using this editor are always semantically sound, as they are governed by the workflow editing engine.
3. Decision support system: This sub-system uses the event log information to guide the user in modelling/editing the care pathways.

FIG. 1 shows the overall workflow and connections of our system. In the following section, we explain in detail the role of each component in the system.

This approach combines the traditional process modelling process with the information from event logs and using the smart process discovery algorithms in assisting the user with interactively modelling the process model. Starting with an empty process model, the user can interactively start adding an activity to the process model. The user first chooses the activity to be added to the process model. Using process discovery/alignment techniques, we learn the best possible position of the activity in the current model. This position is highlighted to the user, by projecting visualizations in the current model to indicate where the activity should be placed. The user has complete control over where to eventually place the activity. Hence the user is essentially modelling the process model, but is always updated with how the actions are reflected in reality (i.e. the event log). The user can either model a complete model, or a sub-model, or a configurable process model.

### Interactive Process Discovery by Synthesizing Sound Free-Choice Workflow Nets Guided by Event Logs

Process discovery algorithms address the problem of discovering process models from event logs. Most discovery algorithms do this automatically by learning process models based on the underlying event data and representing them in the language supported by the algorithm. The representational bias of many of these algorithms doesn't support important properties such as duplicate activities. More importantly, user activity is limited to configuring the parameters of the discovery algorithm and the user has no influence over the actual discovery approach. Hence, user expertise/domain knowledge cannot be utilized during the traditional process discovery approaches. In a setting where the event logs are noisy, or where there are many activities (of which not all may be interesting for the user) the process models discovered by discovery algorithms may be unsound, inaccurate and/or incomprehensible. To overcome the shortcomings of traditional automated process discovery algorithms, we introduce a new approach to interactively discover a process model using the expert knowledge from the user, guided by the event log to assist in decision making. The approach is based on well-known synthesis rules for Petri nets, which guarantee synthesis of any live and bounded free-choice Petri net. The user is guided based on the data from the event log to make informed discovery decisions such as accurate positioning of an activity in the process model, skipping certain activities, adding silent activities, adding duplicate activities, etc.

### INTRODUCTION

Process mining techniques focus on bridging the gap between event logs and business process management. Process mining can be categorized into (1) process discovery, (2) conformance checking and (3) enhancement. Among these, process discovery is highly challenging and is at the spotlight of the research. A large number of discovery algorithms focus on automatically discovering the process model from the event log. The graphical notation used (Petri nets, BPMN models, EPC's, process trees etc) to represent the process model varies from algorithm to algorithm.

The outcome of the discovery algorithm is constrained by the vocabulary of the language used for representing the model. The vocabulary of the modeling language used by the discovery technique determines the representational bias. The representational bias of many discovery algorithms does not include silent and duplicate activities. For example, algorithms such as heuristic miner and inductive miner can not introduce duplicate activities and a miner can not introduce silent transitions. A sound process model guarantees an option to execute each step in the process at least once and the ability to reach the final step (thereby terminating the process) from any valid reachable step. In practical settings, unsound models are often not interesting to the user and are discarded. Ideally the representational bias of the discovery algorithm should limit the search space to only sound models. However, for many discovery techniques this is not the case. For example, algorithms such as a miner and ILP miner) that discover process models as Petri nets do not guarantee the soundness of the discovered model. This is partly because Petri nets have a large expressive power, thereby having complex structural and behavioral properties. Analyzing the behavioral properties (and hence soundenss) of arbitrary Petri nets could be far from trivial and is often not the main focus of the process discovery algorithms. However, a class of Petri nets, called free-choice Petri nets preserve certain behavioral properties that guarantee the soundness.

In this paper we address the issue of discovering a sound process model having a representational bias that is quite expressive allowing for duplicate and silent activities. Process discovery techniques use the four quality dimensions of process mining (fitness, precision, generalization and simplicity) as the guiding principle. Most discovery algorithms explicitly focus on the fitness and/or precision dimensions during discovery. This is logical because both precision and fitness can be calculated entirely based on the event log. The generalization dimension evaluates the degree to which the discovered model is generic, i.e. the extent to which it is coherent with some unseen future behavior. Therefore, it is difficult to quantify the generalization dimension entirely based on the event log and often ignored by many discovery techniques. Finally, the simplicity dimension addresses the complexity of the discovered model. Simplicity is an extremely subjective measure and not taken into consideration by most of the discovery algorithms.

In the interactive process discovery approach presented in this paper, the user discovers the process model incrementally. Since the user has total control over the discovery approach, the user can discover/model the process at a desired complexity level. Therefore, the user can balance simplicity of the model, and to a certain extent—generalization of the model, (among other dimensions) in an efficient way.

Automated process discovery algorithms work well in settings where the event log contains all the necessary (e.g. noise free, complete) information required by the algorithm and the language of the underlying model is the same as the language of the models discovered by the discovery algorithm. However, in many real world scenarios this is not the case. Therefore it is imperative to let the user influence the discovery approach directly. In this paper, we provide such an approach that allows interactive process discovery to discover a class of free choice workflow nets. FIG. 1 shows the high level overview of our complete approach. As a first step, an interactive process editor is developed which allows the user to model sound free choice workflow nets. The formal semantics of the editor are based on the three synthesis rules from. These synthesis rules guarantee construction of all possible free choice nets. Furthermore, soundness is always preserved as the class of nets discovered is free choice Petri nets.

The interactive editor is further enhanced with process mining capabilities. The information from event log is used to guide the user in modeling/discovering the process. The user can make informed decisions about where to place a particular activity, depending on the insights gained from the event log. The interactive approach gives the user total control over process discovery. Besides the objective parameters, our approach allows the user to include the subjective metrics, such as net complexity, which cannot be utilized in traditional automated process discovery techniques.

The remainder of the paper is structured as follows. In Section 2 and Section 3, we provide a literature review of related work and the preliminaries respectively. In Section 4 and Section 5 we discuss the concepts and elements central to our approach. In Section 6 we provide an overview of the implementation. In Section 7 we evaluate our approach based on a real life event log followed by conclusion and future research direction in Section 8.

### Related Work

Process mining as a field has matured over the recent years. The lion's share of research in the process mining community focusses on process discovery techniques. In this section, we first review the state of the art process discovery techniques followed by an overview of Petri net synthesis techniques, interactive net synthesis techniques and usage of domain knowledge in process discovery.

Discovery algorithms which use the information from the event log about ordering relations in activities and their frequencies such as the a miner and heuristic miner are sensitive to noise. Alongside these, the techniques that use semantic approach for discovery such as the ILP miner, state based region miner, and language based region miner do not guarantee soundness. Genetic discovery techniques such as have excessive run times and do not give any guarantees on any quality criterion. Inductive Miner can discover only block structured process models and cannot discover duplicate activities. Our approach differs from all these automated process discovery techniques in multiple ways: The free choice workflow nets generated by our approach are always sound, since we use the synthesis rules kit which guarantees soundness. In our approach, the user has control over the discovery (modeling) process, therefore addition of constructs such as duplicate activities, invisible activities, (self-)loops etc. are all allowed as deemed appropriate by the user. Also, noisy information could be ignored based on the event log information presented to the user. A user who is a domain expert can use the domain knowledge in order to overcome an incomplete event log.

The technique of synthesizing Petri nets from smaller nets is not new, and has been well-researched for over two decades. Our main focus is not to suggest new synthesis rules, but to develop an approach that allows the possibility of using synthesis rules in an interactive way. The state based region theory tool in provides a way of synthesizing Petri nets. However, this approach does not allow user interaction and it does not guarantee soundness of the discovered Petri net. In some examples, a way of interactively synthesizing livebounded Petri nets uses the knitting technique. However, the completeness and soundness of these rules is not guaranteed.

Of late, there has been an interest in using domain knowledge along with the event logs for process discovery. However the domain knowledge is usually represented in some pre-defined constructs or some sort of ‘rules’ which are used as input during the process discovery. The language used to represent the domain knowledge severely limits the expressiveness of the domain expert. In our approach, the user has total control over the discovery phase, and can intuitively use the domain knowledge along with event logs to interactively visualize the process model. It is known to provide a way for the user to include domain expertise in the a algorithm. However, this approach is tied to the underlying a algorithm and does not guarantee the soundnes of the discovered net. It is known to provide a way of introducing domain knowledge specific to an activity which is then used by the a algorithm, rather than an interactive exploration of the process model.

### Preliminaries

This section introduces some basic definitions and background of the concepts used in the interactive process discovery approach. We start with the introduction of bags, followed by Petri nets, workflow nets and free-choice nets. The definitions corresponding to each type of net are sub-classified based on structural and behavioral properties. Next, we discuss the synthesis rules for free-choice nets, followed by the definition of the event logs.

### Bags

A bag over some set S is a function from S to the natural numbers that assigns only a finite number of elements from S a positive value. For a bag B over set S and s E S, B(s) denotes the number of occurrences of s in B, often called the cardinality of s in B. Note that a finite set of elements of S is also a bag over S, namely the function yielding 1 for every element in the set and 0 otherwise. The set of all bags over set S is denoted B(S). We use brackets to explicitly enumerate a bag and superscripts to denote cardinalities. Bag B is a subbag of bag B′, denoted B≤B′, iff, for all s∈S, B(s)≤B′(s).

### Petri Nets

### Structure

Definition 1 (Petri net). A Petri net N is a tuple (P, T, F) such that:
P is a finite set of places,
T is a finite set of transitions such that P∩T=Ø, and
F⊆(P×T)∪(T×P) is the set of arcs, also called the flow relation.
   Definition 2 (Node, preset, postset). Let N=(P, T, F) be a Petri net. Any n∈P∪T is a node, that is, any place or any transition is a node. For any node n∈P∪T, the preset of n in N, denoted N⋅n, is the set of all nodes that have an arc to n, that is, N⋅n={n′|(n′, n)∈F}. Likewise, for any node n∈P∪T, the postset of n in N, denoted n N⋅, is the set of all nodes that have an arc from n, that is, n N⋅={n′|(n, n′)∈F}.

In case the net N is clear from the context, we typically omit it from these notations and use ⋅n instead of N⋅n etc. We also extend the notions of preset and postset to sets of nodes, which results in sets of nodes. If X⊆P∪T, then N⋅X is defined as the set union of the respective presets, that is, N⋅X=∀_{n∈X}{N⋅n}. Likewise, X N⋅=∀_{n∈X}{n N⋅}. It should be noted that in our use of notation, we consider that the preset (or postset) of a set of nodes results in a set of nodes as the output, instead of a bag of nodes.

Definition 3 (Strongly connected Petri net).

A Petri net N=(P, T, F) is called strongly connected if and only if ∀_{x,y∈P∪T}(x, y)∈F*, where F* is the reflexive transitive closure of F.

Definition 4 (Net projection).

Let N=(P, T, F) be a Petri net, let P′⊆P be a subset of places, and let T′⊆T be a subset of transitions. The projection of net N on P′ and T′, denoted N is defined as the net N′=(P′, T′, F′) where F′=F∩((P′×T′)∪(T′×P′)).

### Definition 5 (S-net, S-component, S-coverability).

Let N=(P, T, F) and N′=(P′, T′, F′) be two Petri nets such that N′=N↓_{P′}^{T′. The net N′ is called an S-net if and only if for all t′∈T′ it holds that |N′⋅t′|=}1=|t′N′⋅|, that, every transition has a single place in its preset and a single place in its postset. The S-net N′ is called an S-component of net N if and only if N′ is strongly connected and for all p′∈P′ it holds that N⋅p′∪p′N⋅⊆T′. The net N is called S-coverable if and only if for every place p∈P there exists an S-component that contains p.

Definition 6 (Incidence matrix).

Let N=(P, T, F) be a Petri net. The incidence matrix of N, denoted I(N), is the matrix∈(P×T)→{1, 0,−1} such that

$$I ( N ) ( p , t ) = \{ \begin{matrix}- 1 , & if ( p , t ) ∈ F and ( t , p ) ∉ F ; \\ 1 , & if ( p , t ) ∉ F and ( t , p ) ∈ F ; \\ 0 & otherwise .\end{matrix}$$

The column vector P→{−1, 0, 1} of I(N) is associated to a transition in the net N. Similarly, the row vector T→{−1, 0, 1} of I(N) is associated to a place in the net N.

### Behavior

### Definition 7 (Marking).

Let N=(P, T, F) be a Petri net. A marking M of N is a bag over the places of N, that is, M∈B(P).

A marking M is typically represented as a collection M(p) of tokens for every place p. Removing a token from p then corresponds to removing one occurrence of p for M, and adding a token to p corresponds to adding one occurrence of p to M.

Definition 8 (Transition enabledness, firing).

Let N=(P, T, F) be a Petri net, let M be a marking of N, and let t∈T be a transition of N. Transition t is enabled in M, denoted M t→, if and only if ⋅t≤M. An enabled transition may fire, which leads to a new marking M′, where M′=M−⋅t+t⋅, that is, in the new marking, a token is first removed from every place in the preset of t and a token is then added to every place in the postset of t. This firing is denoted M t→M′.

Corollary 1 (Number of tokens does not change in S-component).

Let N=(P, T, F) be a Petri net, and let N′=(P′, T′, F′) be an S-component of N. The accumulated number of tokens in P′ does not change. Proof. This is straightforward to check, as any transition in T′ removes one token and adds one token.

Definition 9 (Reachable marking).

Let N=(P, T, F) be a Petri net and let M and M′ be markings of N. Marking M′ is called reachable from marking M, denoted M*→M′, if and only if:
either M=M′ or
there exists a marking M″∈B(P) and a transition t∈T such that M t→M″ and M″*→M′.

We use R(N,M) to denote the set of markings reachable from marking M in Petri net N: R(N,M)={M′|M′∈B(P)∧M*→M′}.

Definition 10 (Liveness, deadlock-freedom).

Let N=(P, T, F) be a Petri net, and let M be a marking of N. A transition t∈T is called live in M if and only if for all M′∈R(N,M) there exists a M″∈R(N,M′) such that M″ t→, that is, transition t can get enabled from any marking reachable from M. The net N is called live in M if and only if all its transition are live in M. The net (P, T, F) is deadlock-free in M if and if for all M′∈R(N,M) there exists t∈T, such that M′ t→, that is, every reachable marking enables at least one transition.

Definition 11 (Boundedness, safeness).

Let N=(P, T, F) be a Petri net, and let M be a marking of N. A place p∈P is called k-bounded in M, where k is a natural number, if and only if for all M′∈R(M) it holds that M′(p)≤k, that is, place p never holds more than k tokens. A place p∈P is called bounded in M if and only if there exists a natural number k such that p is k-bounded in M. A place p∈P is called unbounded in M if and only if it is not bounded in M. A place p∈P is called safe in M if and only if it is 1-bounded in M. The net N is called bounded in M if and only if all its places are bounded in M, it is called unbounded in M otherwise. The net N is called safe in M if and only if all its places are safe in M.

### Workflow Nets

### Structure

Definition 12 (Workflow (WF) net).

A Workflow net (or WF net) W is a tuple (P, T, F, i, o, [symbol] , ⊥) such that:
(P, T, F) is a Petri net,
i∈P∧⋅i=Ø, that is, i is a place with an empty preset,
o∈P∧o⋅=Ø, that is, o is a place with an empty postset,
[symbol] , ⊥∈T and i⋅= [symbol] ∧⋅o=⊥, and
∀n∈P∪T(i, n)∈F*∧(n, o)∈F*, that is, every node n is on some path from i to o.

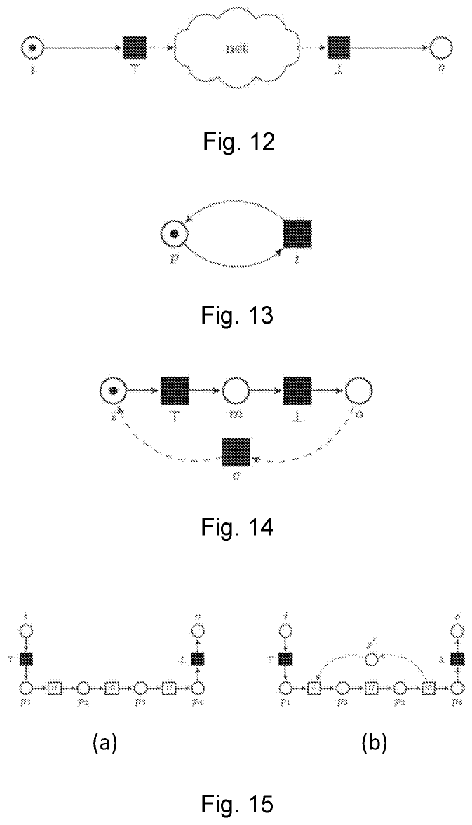

A WF net (P, T, F, i, o, [symbol] , ⊥) is also a Petri net (P, T, F). For this reason, we allow ourselves to use a WF net where a Petri net is expected. FIG. 12 shows an example WF net. Note that we build upon the definition of workflow nets from, by including new T and ⊥ transitions. These transitions are added in order to incrementally maintain the structure of workflow upon using the synthesis rules, as would become evident in the sections to follow.

Definition 13 (Short-circuited WF net).

Let W=(P, T, F, i, o, [symbol] , ⊥) be a WF net. The short-circuited net of W, denoted C(W), is the Petri net (P, T∪{c}, F∪{(c, i), (o, c)}), where c∉P∪T.

In the remainder of this paper, we assume that the short-circuiting transition is the same for all WF nets, and that it is always called c.

Definition 14 (WF Net projection).

Let W=(P, T, F, i, o, [symbol] , ⊥) be a WF net, let P′⊆P be a subset of places such that {i, o}⊆P′, and let T′⊆T be a subset of transitions such that { [symbol] ,⊥}⊆T′ and the net C((P, T, F)↓_{P′}^{T′}) is strongly connected. The projection of WF net W on P′ and T′, denoted W↓_{P′}^{T′} is defined as the WF net W′=(P′, T′, F′, i′, o′, [symbol] , ⊥) where (P′, T′, F′)=(P, T, F)↓_{P′}^{T′}, i′=i, o′=o, [symbol] ′= [symbol] and ⊥′=⊥.

FIG. 12 shows an example workflow net structure. Place i is the only source place and place o is the only sink place. T and ⊥ are always the first and last transitions respectively.

Behavior Definition 15 (Initial marking, final marking, reachability).

Let W=(P, T, F, i, o, [symbol] , ⊥) be a WF net. The initial marking of W is the marking [i], the final marking is the marking [o], and the reachable markings of W are all markings that are reachable from the initial marking, that is, R(W, [i]).

As the initial marking is implicit in a WF net, we typically write R(W) instead of R(W, [i]).

### Definition 16 (Soundness).

Let W=(P, T, F, i, o, [symbol] , ⊥) be a WF net. WF net W is called sound if the following three conditions hold:

1. For every marking M∈R(W) it holds that M*→o, that is, from every marking reachable from the initial marking, the final marking can be reached.

2. For every marking M∈R(W) such that [o]≤M it holds that [o]=M, that is, the final marking is the only reachable marking that includes place o.

3. For every transition t E T there is a M∈R(W) such that M t→, that is, every transition can be fired in some reachable marking.

Theorem 1 (Soundness vs. liveness and boundedness).

Let W=(P, T, F, i, o, [symbol] , ⊥) be a WF net. The net W is sound if and only if the net C(W) is live and bounded in [i].

### Free-Choice Nets

Structure Definition 17 (Free-choice (FC) net).

Let N=(P, T, F) be a Petri net. Petri net N is called a free-choice net (or a FC net) if and only if for every two places p, p′∈P either p⋅∩′⋅=Ø or p⋅=p′⋅.

### Behavior Definition 18 (Well-formedness).

Let N=(P, T, F) be a FC net. The net N is called well-formed if and only if a marking M∈B(P) exists such that N is live and bounded in M.

Corollary 2 (Sound FC WF nets correspond to well-formed nets).

Let W=(P, T, F, i, o, [symbol] , ⊥) be a sound FC WF net. Then the Petri net C(W) is well-formed.

Proof. As W is a sound WF net, we know that the Petri net C(W) is live and bounded in [i].

FIG. 13 shows an atomic net which serves as the initial net for free choice net synthesis using synthesis rules.

Theorem 2 (Well-formedness induces S-coverability).

Let N=(P, T, F) be a well-formed FC net. Then the net N is S-coverable.

Theorem 3 (Liveness and deadlock-freedom).

Let W=(P, T, F, i, o, [symbol] , ⊥) be a bounded WF net. Then C(W) is live in [i] if and only if it is deadlock-free in [i].

Proof. Follows the fact that a short-circuited WF net is strongly connected.

Corollary 3 (Sound FC WF nets are safe).

Let W=(P, T, F, i, o, [symbol] , ⊥) be a sound FC WF net. Then the Petri nets C(W) and (P, T, F) are safe in marking [i].

Proof. As W is a sound WF net, we know that the Petri net C(W) is well-formed, and is hence S-coverable. As the marking contains only a single token in place i, and as every place p is covered by some S-component, place p cannot contain more than one token in R(C(W), [i]). As R((P, T, F), [i])⊆R(C(W), [i]), place p cannot contain more than one token in R((P, T, F), [i]).

### Synthesis Rules for FC Nets

In this section, we discuss the three synthesis rules, as they form the skeleton of our approach. It has been proven that these rules are complete and can deduce any sound free-choice net. The synthesis rules are valid for all free choice nets. The starting net for synthesis rules is a live and bounded atomic net containing only one place and transition as shown in FIG. 13.

The abstraction rule allows expansion of a net by adding a new place p_{new} and a new transition t_{new}, between a set of transitions R and set of places S. The abstraction rule can be formally defined as:

### Definition 19. (Abstraction Rule ψ_{A})

Let N=(P,T,F) and N′=(P′, T′, F′) be two FC nets. N′ is synthesized from N, i.e. (N,N′)∈ψ_{A} if and only if:
P′=P∪{p} (where p∉P)
T′=T∪{t} (where t∉T)
R×S⊆F such that R⊆T and S⊆P
F′=F\(R×S) ∪ ((R χ {p}) ∪ ({p}×{t}) ∪ ({t}×S))

Next, we discuss the two linearly dependent synthesis rules. A linearly dependent rule allows introduction of a new linearly dependent place or transition in a net. We begin by first introducing linearly dependent transitions and places, followed by the definitions of the rules. From basic linear algebra we know that a column c belonging to a matrix M is linearly dependent if and only if c can be expressed as some linear combination of the columns in M.

### Definition 20. (Linearly Dependent Nodes)

Let N=(P, T, F) be a Petri net and I(N) be the incidence matrix of N. Let Q be the set of all rational numbers. A place p of N is linearly dependent if there exists a vector Λ: P→Q such that Λ(p)=0 and Λ⋅I(N)=p. Similarly, a transition t of N is linearly dependent if there exists a vector Λ: T→Q such that Λ(t)=0 and Λ⋅I(N)=t. The linearly dependent place p (transition t) is non-negatively linearly dependent if moreover Λ≥0.

Following the definition of linearly dependent nodes, we now discuss the two linearly dependent rules, which allow addition of a new transition or a new place to the net.

### Definition 21. (Linear Dependent Transition Rule ψ_{T})

Let N=(P,T,F) and N′=(P′, T′, F′) be two FC nets. N′ is synthesized from N, i.e. (N,N′)∈ψT if and only if:
P′=P
T′=T∪{t}, where t∉T
F′=F∪^{˜}F, where ^{˜}F⊆((P×{t})∪({t}×P))
t is a linearly dependent transition of N′
N′⋅t∪tN′⋅≠Ø

### Definition 22. (Linear Dependent Place Rule ψ_{P})

Let N=(P,T,F) and N′=(P′, T′, F′) be two FC nets. N′ is synthesized from N, i.e. (N,N′)∈ψP if and only if:
T′=T
P′=P∪{p}, where p∈P
^{˜}F=F∪^{˜}F, where ^{˜}F⊆(({p}×T)∪(T×{p}))
p is a linearly dependent place of N′
N′⋅p∪pN′⋅≠Ø
   Theorem 4 (Synthesis rules preserve well-formedness).

Let N=(P, T, F) and N′=(P′, T′, F′) be two FC nets such that (N,N′)∈ψ_{A}∪ψ_{P} ∪ψ_{T}. Then N′ is well-formed if N is well-formed.

Observation An interesting observation on these synthesis rules is that the presets of transitions can only grow, while the postset of places can also only grow. The rule ψ_{A} allows for a transition to reduce the size of its postset to 1 and for a place to reduce the size of its preset to 1, but there is no rule that can remove an arc from a place to a transition.

### Event Logs

Let A be the set of all process activities.

Definition 23 (Event log).

An event e is the occurrence of an activity: e∈A. A trace σ is a (possibly empty) sequence of events: σ∈A*. We denote an empty trace as ε, and use [symbol] σ₁, . . . , σₙ [symbol] to denote a non-empty trace σ, where n is the length of σ, that is, n=|σ|. Note that for every 1≤i≤|σ|, σᵢ is an event. An event log L is a non-empty bag of traces, that is, L∈B(A*).

Note that the same trace may appear multiple times in an event log. Each trace corresponds to an execution of a process, i.e., a case.

### Approach

In this section we discuss the approach used in order to enable interactive process discovery using the synthesis rules.

### Synthesis Rules for FC WF Nets

The synthesis rules allow synthesis of all free-choice nets. However, in our approach we are only interested in sound free-choice workflow nets. Hence we first define the initial net for our approach followed by the derived version of three synthesis rules specific to sound FC WF nets.

Definition 24 (Initial sound FC WF net W₀).

The initial sound FC WF net W0 is the WF net: W0=({i, m, o}, { [symbol] ,⊥}, {(i, [symbol] ), ( [symbol] ,m), (m,⊥), (⊥, o)}, i, o, [symbol] , ⊥). (See FIG. 14)

Definition 25 (Abstraction rule ψ′_{A} for FC WF nets).

Let W=(P, T, F, i, o, [symbol] , ⊥) and W′=(P′, T′, F′, i′, o′, [symbol] , ⊥) be two FC WF nets. W′ can be synthesized from W, denoted (W,W′)∈ψ′_{A}, if and only if (C(W),C(W′))∈ψ_{A}.

Note that abstraction rule allows addition of a new place and a new transition, between a (set of) transition(s) and place(s). However, it is not possible to use the abstraction rule between the transition ⊥ and the sink place o, as the resulting net would no longer be a workflow net as defined in Definition 12.

FIG. 4 shows a short-circuited FC Net which can be transformed into a FC WF by removing the transition c. This net (without c) serves as the initial net Wₒ of our approach.

Theorem 5 (Abstraction rule ψ′_{A} preserves soundness).

Let W=(P, T, F, i, o, [symbol] , ⊥) and W′=(P′, T′, F′, i′, o′, [symbol] , ⊥) be two FC WF nets such that (W,W′)∈ψ′ A. Then the net W′ is sound if the net W is sound.

Proof. To show that W′ is sound, we need to show that (P′, T′, F′) is live and bounded in [i′].

### Boundedness

The net W is sound, hence C(W) is live and bounded in [i], hence C(W) is well-formed. C(W′) is also well-formed, thus making it S-coverable, and hence bounded for any marking M′, and hence bounded in [i′].

### Liveness

As C(W) is bounded in [i′] and strongly connected, if suffices to show that it contains no deadlocks in [i′], which follows from the facts that (1) C(W) does not contain deadlocks in [i] and (2) by construction this rule cannot introduce new deadlocks.

Definition 26 (Linearly dependent nodes for sound FC WF nets).

Let W=(P, T, F, i, o, [symbol] , ⊥) be a sound FC WF net. Let C(W) be the short circuited version of W. Recall that I(C(W)) denotes the incidence matrix of net C(W). A place p∈P is called linearly dependent in W if and only if there exists a vector V∈P→{0, 1} such that V (p)=0 ∧V⋅I(C(W))=p. A transition t∈T is called linearly dependent in W if and only if there exists a vector V∈T→{0, 1} such that V (t)=0 ∧I(C(W)) ⋅V=t.

Theorem 6 shows that we can consider the linear dependencies within the set {0, 1} instead of the set of rational numbers Q.

Theorem 6 (Linear dependencies span over the set {0, 1}).

Let N=(P, T, F) be a well-formed FC net, and let t∈T be a non-negative linearly dependent transition in N. Let I(N) denote the incidence matrix of N, then there exists a vector V∈T→{0, 1} s.t. V (t)=0 ∧I(N)⋅V=t.

Proof. To show that it suffices to have the set of linear dependencies as {0, 1}, we need to show that there exists a minimal semi-positive T-invariant which has weights either 0 or 1. As N is well-formed it is T-coverable. Hence there exists a T-component that covers t, which induces a semi-positive T-invariant X that covers t. By construction, (X−t)+V is also a semi-positive T-invariant. Hence (X−t)+V is the sum of minimal semi-positive invariants. At least one of these minimal semi-positive invariants covers (X−t). Take such a minimal semi-positive invariant (X−t)+Y. Therefore, Y (t)=0 ∧I(N)⋅Y=t. As Y induces a T-component, all the weights in Y are equal (i.e. 1, and all the other weights are 0). In a similar way, we can show this for linearly dependent places, as the net N is also S-coverable.

Definition 27 (Linearly dependent place rule ψ′_{P} for FC WF nets).

Let W=(P, T, F, i, o, [symbol] , ⊥) and W′=(P′, T′, F′, i′, o′, [symbol] , ⊥) be two FC WF nets. W′ can be synthesized using ψ′_{P}, denoted (W,W′)∈ψ′_{P}, if and only if:

1. (C(W),C(W′))∈ψ_{P}

2. p′∈P′\P is not linearly dependent on a set of places that includes i′

The second condition of Definition 27 limits the synthesis to only the sound FC WF nets, by avoiding linearly dependent places which could result in deadlocks. The need for this condition is elaborated in Theorem 7.

Theorem 7 (Deadlock avoidance in FC WF nets after using ψ′_{P}).

Let W=(P, T, F, i, o, [symbol] , ⊥) be a sound FC WF net. If a new place p is added W (such that p/∈P) that causes a deadlock in W then W becomes unsound after introduction of p.

Proof. A siphon is defined on a set of places S, such that: ⋅S⊆S⋅. If S is non-empty, then it is a proper siphon. A trap is defined as a set of places S, such that: S⋅⊆•S. If S is non-empty, then it is a proper trap. According to commoners theorem, a free choice system is live if and only if every proper siphon includes an initially marked trap. We know that a short-circuited net C(W) is live if the source place of the W, i is marked. If introduction of a new place in the net results in a siphon §, such that i/∈§, then according to Commoners theorem the newly added place should be marked. If the new place is not marked, it would result in a deadlock in the FC WF net, and hence the net would no longer be sound (i.e. not well-formed).

Hence every new place added to FC WF net must not result in any siphon in the net W.

### Corollary 4.

A new place p containing the same inputs and outputs cannot be added to a FC WF net using ψ′_{P}.

Proof. If p has the same input and output transitions, then we have ⋅p=p⋅. Therefore, p forms a set which is a proper siphon and a proper trap because ⋅{p}⊆{p}⋅ and {p}⋅⊆⋅{p}. Since p is newly added, it is not marked, and hence by Theorem 7, p is invalid as an unmarked p would result in a deadlock.

Theorem 8 (Linearly dependent place rule ψ′_{P} preserves soundness).

Let W=(P, T, F, i, o, [symbol] , ⊥) and W′=(P′, T′, F′, i′, o′, [symbol] , ⊥) be two FC WF nets such that (W,W′)∈ψ′P. Then the net W′ is sound if the net W is sound.

Proof. To show that W′ is sound, we need to show that (P′, T′, F′) is live and bounded in [i′].

FIG. 15 is an example demonstrating Theorem 7. FIG. 15(a) shows an example sound FC WF net, with source place i and sink place o. FIG. 15(b) shows an example of adding a linearly dependent place p′. An unmarked p′ results in a deadlock.

### Boundedness

The net W is sound, hence C(W) is live and bounded in [i], hence C(W) is well-formed. Therefore, C(W′) is also well-formed, hence Scoverable, hence bounded for any marking M′, and hence bounded in [i′].

### Liveness

As C(W′) is bounded in [i′] and strongly connected, it suffices to show that it contains no deadlocks in [i′]. The second condition of Definition 27 guarantees that Theorem 7 is not violated, ultimately guaranteeing that the synthesized net is deadlock free. Hence, C(W′) contains no deadlocks in [i′], hence it is live in [i′].

Definition 28 (Linearly dependent transition rule ψ′_{T} for FCWF nets).

Let W=(P, T, F, i, o, [symbol] , ⊥) and W′=(P′, T′, F′, i′, o′, [symbol] , ⊥) be two FC WF nets. W′ can be synthesized using ψ′_{T}, denoted (W,W′)∈ψ′_{T}, if and only if (C(W),C(W′))∈ψ_{T}. It should be noted that the newly added transition using ψ′_{T} is linearly dependent on all [symbol] ,⊥ and the short circuited transition c in C(W) or none of the transitions [symbol] ,⊥ and c in C(W). This follows from the definition of WF nets in Definition 12.

Theorem 9 (Linearly dependent transition rule ψ′_{T} preserves soundness).

Let W=(P, T, F, i, o, [symbol] , ⊥) and W′=(P′, T′, F′, i′, o′, [symbol] , ⊥) be two FC WF nets such that (W,W′)∈ψ′_{T}. Then W′ is sound if the net W is sound.

Proof. To show that W′ is sound, we need to show that C(W′) is live and bounded in [i′].

### Boundedness

The net W is sound, hence C(W) is live and bounded in [i], hence C(W) is well-formed. Hence, C(W′) is also well-formed, hence S-coverable, hence bounded for any marking M′, and hence bounded in [i′].

### Liveness

As C(W′) is bounded in [i′] and strongly connected, it suffices to show that it contains no deadlocks in [i′], which follows from the facts that (1) C(W) contains no deadlocks and (2) the set of reachable markings does not change by introducing the new transition t′, as the marking after t′ has fired could already be reached by firing the transitions it was linearly dependent on.

In the case of workflow nets, the linearly dependent transition rule allows addition of a new transition which essentially is a choice (or a loop back) to a (set of) transition(s). The linearly dependent place rule allows addition of a new place, which can be used to introduce concurrency in the system. The linearly dependent place (transition) is a redundant place (transition) such that there are other places (transitions) which together have the same incoming and outgoing transitions (places).

The starting net (W₀) for our approach is shown in FIG. 4. The user interaction and synthesis is allowed only between the transitions [symbol] and ⊥. Place i is the only source and marked place in the net, and place o is the only sink place in the net. C(Wₒ) can be obtained by adding a transition to the net, starting from o and ending with i would make the system live and bounded.

### Theorem 10.

Any sound FC WF net W′ can be derived from the initial net Wo (FIG. 14).

Proof. We know that any FC net can be deduced using the original synthesis rules. The new synthesis rules introduced in this paper, (Definition 32, Definition 33 and Definition 34), for FC WF nets are derived from the original rules, to preserve the soundness properties of FC WF nets. Hence, the new rules, which differ from the original rules only in the sense that they restrain the old rules to assure soundness, are sufficient to derive any sound FC WF net.

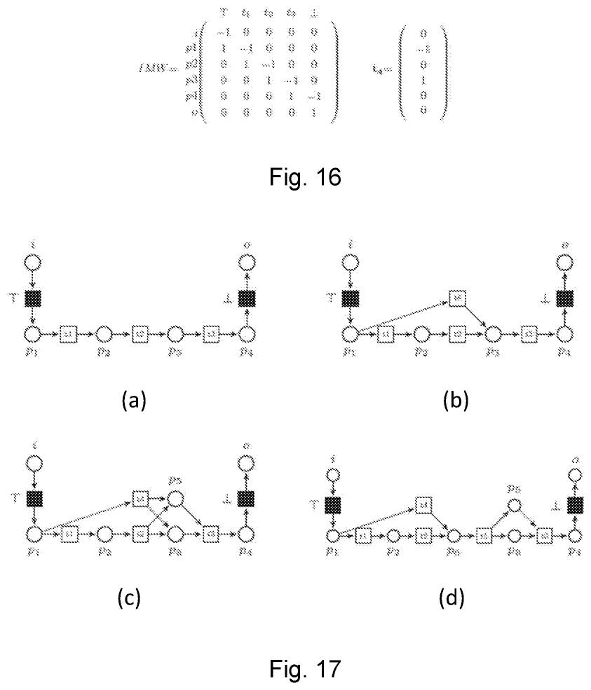

FIG. 17 shows the applications of all the synthesis rules for sound FC WF nets. The linear dependencies (both transition and place) can be calculated using the incidence matrix. As evident from FIG. 16, t₄=t₁+t₂. FIG. 17b is synthesized from FIG. 17a by adding a new linearly dependent transition t₄ using ψ′_{T}. FIG. 17b is further synthesized into FIG. 17c by adding a new linearly dependent place ₚ₅ using ψ′_{P}. FIG. 17d shows the application of abstraction rule (ψ′_{A}) for FC WF nets to the net from FIG. 17c.

FIG. 16 shows an incidence matrix of FIG. 17a and linearly dependent transition t₄ of FIG. 17b.

FIG. 17 shows examples showing the application of all three synthesis rules (ψ′_{T}, ψ′_{P}, ψ′_{A}) for sound FC WF nets. FIG. 17(a) shows an example sound FC WF net W. FIG. 17(b) shows adding t₄ to the net from 7(a) using ψ′_{T}. FIG. 17(c) shows adding p₅ to the net from 17(b) using ψ′_{P}. FIG. 17(d) shows adding p₆ and t₅ to the net from 17(c) using ψ′_{A}.

### 4.2 Synthesis Space

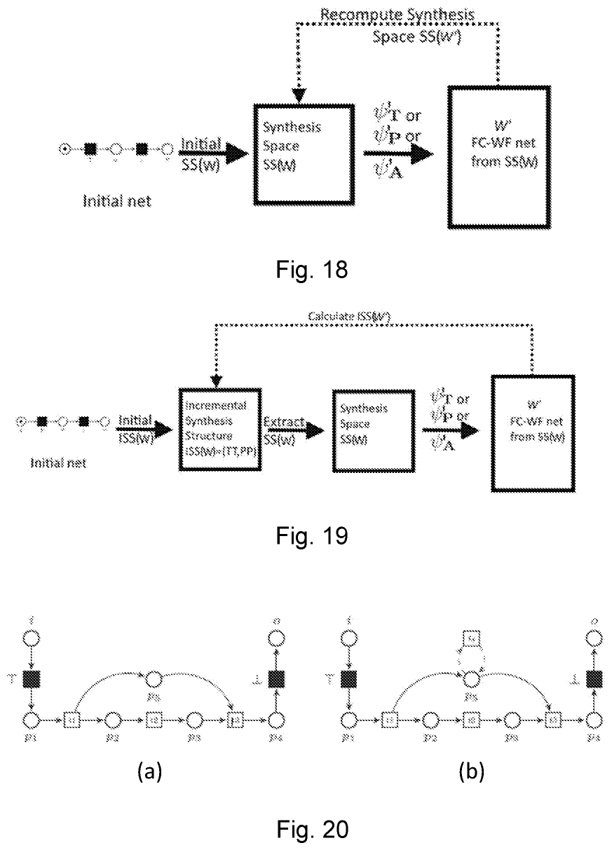

FIG. 18 provides a high level conceptual overview of the steps followed in our approach. Although it is possible to synthesize any sound FC WF net using the synthesis rules, (as shown in Theorem 10), they cannot be used out of the box for sound FC WF net synthesis in a real-time setting. In order to overcome this, we introduce the so called synthesis space, which is the central component of our approach and which contains all the nets that could be synthesized from any net using ψ′_{A}, ψ′_{T} or ψ′_{P}. Formally, the synthesis space is defined as:

### Definition 29 (Synthesis Space SS).

Let W=(P, T, F, i, o, [symbol] , ⊥) be a sound FC WF net, and let FWN be the universe of sound FC WF nets. The synthesis space SS(W)=SSA(W)∪SSP(W)∪SST (W), such that:
SSA(W)={W′∈FWN|(W,W′)∈ψ′_{A}},
SST(W)={W′∈FWN|(W,W′)∈ψ′_{T}},
SSP(W)={W′∈FWN|(W,W′)∈ψ′_{P}}

It should be noted that SSA(W), SSP (W) and SST (W) are all disjoint for a FC WF net W. This is evident from the fact that SSA(W) synthesizes the net by using ψ′_{A}, thereby adding a new transition and a new place to W, whereas SSP (W) and SST (W) synthesizes the net by adding a new place (using ψ′_{P}) or a new transition (using ψ′_{T}) respectively.

FIG. 18 shows an overview of the approach. The synthesis space contains all the nets which can be synthesized from a particular net. Once a net from synthesis space has been selected by the user, the synthesis space is re computed corresponding to the newly selected net.

At any given point, the synthesis space contains all the possible nets that could be synthesized using either ψ′_{A}, ψ′_{P} or ψ′_{T} once. From the synthesis space (SS), starting with a sound FC WF net W the user can select any synthesized sound FC WF net W′. However, upon selecting a synthesized net, the synthesis space would be needed to be re-computed corresponding to the new net. For large nets, this task is far from trivial and could be computationally challenging. This is especially the case for the linear dependency rules as they are nonlocal, and require solving an ordinary system of linear equations to check the linear dependency of the node with respect to the net. Clearly recomputing the synthesis space from scratch every time upon selection of a net would be expensive, inefficient and therefore undesirable in an interactive setting. In order to overcome this, we introduce an incremental approach of re-calculating the synthesis space.

### 4.3 Incremental Synthesis Structure

In order to incrementally update the synthesis space after application of any synthesis rule (ψ′_{A}, ψ′_{T}, ψ′_{P}) to a sound FC WF net, we introduce a new structure called the ‘incremental synthesis structure’. Incremental synthesis structure is an intermediate structure which is used to extract the synthesis space. The application of abstraction rule (ψ′_{A}) is straight-forward as it is a local rule. That is, in order to use ψ′_{A}, it suffices to look only at the set of transitions and places between which the new place and transition should be added in the net. Therefore, for any synthesized net, all the possible applications of ψ′_{A} can be easily and efficiently computed. Hence there is no need to maintain any specific structure with regards to the SSA(W). On the contrary, calculating the linear dependencies to apply ψ′_{P} or ψ′_{T} in a synthesized net is far from trivial and would be computationally very expensive. Therefore we maintain all the possible applications of linear dependencies with respect to every net in the incremental synthesis structure. After synthesizing a net using any rule, the existing linear dependencies in the incremental synthesis structure are updated to reflect the linear dependencies corresponding to the new net. Furthermore, newer linear dependencies introduced in the new net are also added to the incremental synthesis structure. We prove that the incremental synthesis structure is correct and complete to synthesize any net using ψ′_{P} or ψ′_{T}. FIG. 19 provides an overview of the role of the incremental synthesis structure in our approach. We begin by providing the formal definition of the incremental synthesis structure (ISS).

FIG. 19 shows an overview of the approach. The incremental synthesis structure contains all the possible linear dependencies that could be added to a net. The linear dependencies that result in sound FC-WF nets are extracted and the user is presented with all the possible synthesisized nets to choose from. Upon selecting a net, the incremental synthesis structure is recalculated corresponding to the chosen net.

### Definition 30 (Incremental Synthesis Structure ISS).

Let W=(P, T, F, i, o, [symbol] , ⊥) be a sound FC WF net. The incremental synthesis structure ISS(W) is a tuple (TT,PP), where:

1. (a) TT is a set of pairs of sets of places
(b) PP is a set of pairs of sets of transitions

2. (a) (P_{IN}, P_{OUT})∈TT iff⋅t′=P_{IN}, t′⋅=P_{OUT} ∧t′ is linearly dependent in W, such that P_{IN}, P_{OUT} ⊆P∧P_{IN}, P_{OUT}≠Ø.
(b) (T_{IN}, T_{OUT})∈PP iff⋅p′=T_{IN}, p′⋅=T_{OUT} ∧p′ is linearly dependent in W, such that T_{IN}, T_{OUT} ⊆T∧T_{IN}, T_{OUT}≠Ø.

The incremental synthesis structure TT (PP) contains all the linearly dependent transitions (or places) resulting in WF nets. However, it should be noted that, some transitions (or places) from the incremental synthesis structure might be linearly dependent, but could make the net a non-free choice WF net. FIG. 20 shows an example of adding a linearly dependent transition in a net which results in a non-free choice construct. Clearly transition t₄ added to the net is a self loop and hence linearly dependent, however the resultant net from FIG. 20b is no longer a free choice net. As the synthesis kit is valid only for sound FC WF nets, there is an additional step needed to extract the legitimate candidates from the incremental synthesis structure ISS(W) which result only in sound FC WF net.

FIG. 10 shows adding linearly dependent transition to a net resulting in a non-free choice net in FIG. 10 (b). FIG. 10 (a) shows an example FC-WF net. FIG. 10 (b) shows adding a linearly dependent transition t₄ to FIG. 10 (a)

It is important to maintain both ISS(W) and SS(W) for a particular net because we use the pre-existing linear dependencies in ISS(W) to calculate the newer linear dependencies after synthesis. Hence, even if a linearly dependent candidate node in ISS(W) might not result in FC WF net, it might still be needed to compute future linear dependencies of newly added node(s) after synthesis, as would become evident in the upcoming sections. As mentioned previously, SS(W) contains only the sound FC WF nets.

The extraction of legitimate synthesis space for linear dependencies (SST (W) and SSP(W)) from incremental synthesis structure (ISS(W)) is possible and is straight-forward as shown in Theorem 11.

Theorem 11 (Synthesis space for linear dependencies can be extracted from incremental synthesis structure).

Let W=(P, T, F, i, o, [symbol] , ⊥) be a sound FC WF net, and let ISS(W)=(TT,PP) be the incremental synthesis structure of W. Then the synthesis space for linear dependencies SS_{T}(W) and SS_{P}(W) can be extracted from the incremental synthesis structure ISS(W).

Proof. By the definition of incremental synthesis structures (Definition 30), we know that ISS(W)=(TT, PP) contains all the linear dependencies required to generate the synthesis spaces SS_{P}(W) and SS_{T}(W), among many more.

In order to extract the synthesis space for linear dependencies, we first find a sub-set [symbol] and [symbol] from TT and PP as follows:

[symbol]

=∀_{(P}_{_{IN}}_{,P}_{_{OUT}}_{)∈TT}{(P_{IN},P_{OUT}))|∃_{t∈T}⋅t=P_{IN}}

[symbol]

=∀_{(T}_{_{IN}}_{,T}_{_{OUT}}_{)∈PP}{(T_{IN},T_{OUT}))|∃_{P∈P}p⋅=T_{OUT}}

[symbol]

(and [symbol] ) contain all the transitions (and places), each of which when added to W and would result in sound FC WF net. The proof for this is trivial and follows from the definition of FC WF nets. Using [symbol] and [symbol] , SS_{T}(W) and SS_{P}(W) can be computed as follows:

SS_{T}(W)=∀_{(P}_{_{IN}}_{,P}_{_{OUT}}₎ [symbol] {W′|W′ is synthesized form W by adding a transition with input P_{IN} and output P_{OUT} and (W,W′)∈ψ′_{T}}

SS_{P}(W)=∀_{(T}_{_{IN}}_{,T}_{_{OUT}}₎ [symbol] {W′|W′ is synthesized form W by adding a place p′ with input T_{IN} and output T_{OUT} and (W,W′)∈ψ′_{P}}

### 4.4 Incremental Synthesis Structure Updates

Theorem 11 provides a mechanism of extracting the synthesis space from the incremental synthesis structure. The primary reason of introducing the incremental synthesis structure is to assist in incrementally updating the synthesis space after selecting a net from synthesis space. To accomplish this, upon synthesizing a net W′ from W, that is selecting a net from the synthesis space SS(W), we make changes to the incremental synthesis structure ISS(W) in order to derive ISS(W′). This is the calculate ISS(W′) phase of FIG. 19, which is central to our approach. From Theorem 11, we know that we can extract the linearly dependent synthesis space from the incremental synthesis structure. In order to do this however, the incremental synthesis structure should contain all necessary information to required, which allows the extraction of synthesis space. That is, changes made to ISS(W) after selecting a net W′∈SS(W), should guarantee the extraction of SSP (W′) and SST (W).

In this section we discuss the changes in the incremental synthesis structure after the user selects a net from the synthesis space. Every net from synthesis space is basically an application of one of the synthesis rules (ψ′_{A}, ψ′_{P}, ψ′_{T}). After the usage of each synthesis rule, we define the changes made to ISS(W). Furthermore, we prove that the changes made are necessary and sufficient to derive the linearly dependent synthesis space of the new net. We first discuss a function paths B(W, In, Out) which takes as input the FC WF net, and sets of input and output places (transitions) and returns the set of transitions (places) which effectively have the same effect.

### Definition 31. (Paths B(W, In,Out))

Let W=(P, T, F, i, o) be a sound FC WF net. For any pair of sets of places (PIN, POUT), the function P returns the set of sets of transitions which together constitute the same effect as (P_{IN}, P_{OUT}) in C(W). B(W, In,Out) is defined as follows:

B(W, In,Out)={X⊆T|⋅X\X⋅=In∧X⋅\⋅X=Out}

That is, if X∈B(W, PIN, POUT), then firing every t∈X once leads from a marking P_{IN} to marking P_{OUT} in C(W).

Similarly, for any place with input transitions T_{IN} and output transitions T_{OUT}, the function P(W, _{TIN,} T_{OUT}) returns the set of sets of places which together constitute the same effect as (T_{IN}, T_{OUT}) in C(W).

Theorem 12 (At least one additional set of nodes is returned by the paths function for a (non-self loop) linearly dependent node).

Let W=(P, T, F, i, o) be a sound FC WF net. If W′=(P′, T′, F′, i′, o′) is synthesized from W by using ψ′ P by adding a new non-loop place p′ to W, such that W′⋅t′ ≠t′W′⋅. Then P(W′, W′⋅p′, p′W′⋅) contains {p′} and at least one more set of places. Proof. From the definition of linear nodes for FC WF nets (Definition 26) we know that, if p′ is linearly dependent in W′, then there exists a vector V∈P′→{0, 1} such that V (p′)=0∧V ⋅I(C(W′))=p′. Furthermore, from Theorem 6 we know that there exists a minimal semi-positive T-invariant which has weights either 0 or 1. That is, there exists a set of places X (corresponding value 1) forming the minimal semi-positive T-invariant, thereby having the same overall effect as p′ in W′. Similarly it can be shown that for a non-self loop linearly dependent transition t′ added to the net W resulting in W′, there exists is a set of transitions Y which form a minimal semi-positive S-invariant having the same overall effect as t′.

It should be noted that Paths (B(W, In,Out)) operates on the short-circuited net C(W). Therefore, in case of a loop transition, the set of transitions returned by the path function would always contain short circuited transition c. Using the paths function on the net W from FIG. 10a, for transitions {t1} and {t3} as input and output sets respectively, we get the following set of sets as output: B(W, {t1}, {t3})={(p2, p3), (p5)}.

ISS updates for ψ′_{P}

We now discuss the changes in incremental synthesis structure after the usage of a linearly dependent place rule (ψ′_{P}). That is, when the user selects any net from synthesis space, which is a result of a ψ′_{P}.

Definition 32 (Extracting ISS(W′) from ISS(W) after the application of ψ′_{P}).

Let W=(P, T, F, i, o) be a sound FC WF net. Let ISS(N)=(TT, PP) be the incremental synthesis structure corresponding to the net W. Let W′=(P′, T′, F′, i′, o′) be a sound FC WF net synthesized from W by adding a place p′, s.t. (W,W′)∈ψ′_{P}. Then the incremental synthesis structure ISS(W′)=(TT′, PP′) corresponding to W′ can be derived using ISS(W) as follows (note that P′\P={p′}):

$$- {PP}^{′} = PP \\ - {TT}^{′} = \{ ( {p}^{′} ) , ( {p}^{′} ) \} ⋃ \\ {∀}_{( {P}_{IN} , {P}_{OUT} ) ∈ TT} \\ \{ \begin{matrix}( {P}_{IN} , {P}_{OUT} ) & if \text{?} \begin{matrix}\text{?} ⋂ {P}_{IN} = ∅ ⋀ \\ \text{?} ⋂ {P}_{OUT} = ∅\end{matrix} \\ ( {P}_{IN} \vert ⋃ \{ {p}^{′} \} , {P}_{OUT} ) & else if \text{?} \begin{matrix}\text{?} ⋂ {P}_{IN} \neq ∅ ⋀ \\ \text{?} ⋂ {P}_{OUT} = ∅\end{matrix} \\ ( {P}_{IN} , {P}_{OUT} ⋃ \{ {p}^{′} \} ) & else if \text{?} \begin{matrix}\text{?} ⋂ {P}_{IN} = ∅ ⋀ \\ \text{?} ⋂ {P}_{OUT} \neq ∅\end{matrix} \\ ( {P}_{IN} , {P}_{OUT} ) ⋃ & else if \text{?} \begin{matrix}\text{?} ⋂ {P}_{IN} \neq ∅ ⋀ \\ \text{?} ⋂ {P}_{OUT} \neq ∅\end{matrix} \\ ( {P}_{IN} ⋃ \{ {p}^{′} \} , {P}_{OUT} ⋃ \{ {p}^{′} \} ) & \end{matrix} \\ \text{?} \text{indicates text missing or illegible when filed}$$

The ISS(W′) derived using ISS(W) according to Definition 32, should adhere to the definition of incremental synthesis structure (Definition 30). According to Definition 30, ISS(W′) has to adhere to two conditions. Firstly, every node from (TT′, PP′) should be linearly dependent on W′. This is proven using Theorem 13 and Theorem 14. According to the second condition of Definition 30, ISS(W′) should be complete to yield SS_{P}(W′) and SS_{T}(W′). This is proven using Theorem 15.

Theorem 13 (Every place in PP′ extracted using Definition 32 is linearly dependent in W′).

Let W=(P, T, F, i, o) and W′=(P′, T′, F′, i′, o′) be two sound FC-WF nets, s.t. (W,W′)∈ψ′_{P}. Let ISS(N)=(TT, PP) be the incremental synthesis structure corresponding to W. Let ISS(W′)=(TT′, PP′) be the incremental synthesis structure extracted from ISS(W) using Definition 32. Then every place in PP′ is linearly dependent in W′.

Proof. Let p′ be the linearly dependent place added to W to synthesize W′ using ψ′ P (i.e. p′∈P′∧p′∉P). Since p′ itself is a linearly dependent place, the addition of p′ to W cannot result in a change in the existing linear dependencies in PP. That is all the pre-existing linearly dependent places in W are also valid in W′. Therefore if PP contained all the linearly dependent places corresponding to W, these places are also linearly dependent with respect to the net W′.

Theorem 14 (Every transition in TT′ extracted using Definition 32 is linearly dependent in W′).

Let W=(P, T, F, i, o) and W′=(P′, T′, F′, i′, o′) be two sound FC WF nets, s.t. (W,W′)∈ψ′P. Let ISS(N)=(TT, PP) be the incremental synthesis structures corresponding to W. Let ISS(W′)=(TT′, PP′) be the incremental synthesis structure extracted from ISS(W) using Definition 32. Then every transition in TT′ is linearly dependent in W′.

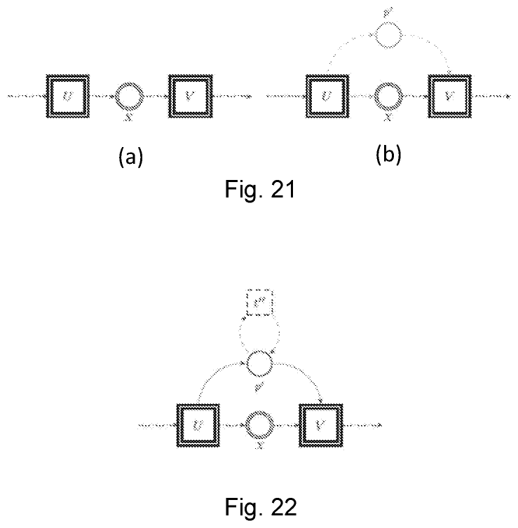

Proof. Let p′ be the linearly dependent place added to W to synthesize W′ using ψ′_{P} (i.e. p′∈P′∧p′∉P). It should be noted that from Corollary 4.1, we know that ψ′ P would not allow synthesis of a self-loop place (i.e. W′⋅p′≠p′W′⋅). Therefore using Theorem 12, we know that since p′ is a non-self loop linearly dependent place, there exists at least one additional set of places ^{˜}P, (^{˜}P∈P(W′, W′⋅p′, p′W′⋅)). Based on the Definition 32, corresponding to each change we split the proof of correctness of extraction for TT′ in five parts, using FIG. 21 as our running example. A double border in FIG. 21 indicates a set of nodes (either set of transitions or set of places).

FIG. 21: shows a running example net fragment for Theorem 14, wherein U and V are sets of transitions and X is a set of places. FIG. 21(a) shows part of a FC WF net. FIG. 21(b) shows an updated part of the FC WF net FIG. 21(a) after the application of ψ′_{P} rule. p′ is the newly added linearly dependent place in with input set of transitions U and output set transition set V.

### Creating a New Self-Loop Transition

Since there is a new place added to the net, there is an additional possibility to add a new transition which starts and ends with the newly added place p′. This transition is a self-loop transition. If this new self-loop transition is added to the net W′, the incidence matrix of W′ (I(W′)) is changed merely by adding a new column corresponding to the self-loop transition, having the values zero in all its cells, and thereby is linearly dependent.

FIG. 22 shows introducing a new linearly dependent transition (self-loop) in the incremental synthesis structure corresponding to the newly added place.

Let [symbol] and [symbol] be the input and output set of places for a non-self loop linearly dependent transition t that can be added to the net W, s.t. ( [symbol] , [symbol] )∈TT. Since \hat{t} is not a self loop and is linearly dependent in W, there exists at least one set of transitions \tilde{T}⊆T (\tilde{T}∈B(W, T_{IN}, T_{OUT})), which have the same overall effect as \hat{t}. That is, starting with a marking P_{IN} if every transition from \tilde{T} is fired exactly once, then the final marking would be P_{OUT}. Formally, ∃\tilde{T}: W⋅\tilde{T}\\tilde{T}W⋅= [symbol] ∧\tilde{T}W⋅\W⋅\tilde{T}= [symbol] .

If P_{IN} ∩\tilde{P}=Ø∧P_{OUT} ∩\tilde{P}=Ø. Recall that \tilde{P} is the set of places which have the same overall effect as p′ in W′. For a linearly dependent transition \hat{t}, if the set of input places P_{IN} and \hat{P} are distinct, and the set of output places P_{OUT} and \hat{P} are distinct then, we can easily see that:

W′⋅\tilde{T}\\tilde{T}W′⋅= [symbol] ∧

\tilde{T}W′⋅\W′⋅\tilde{T}= [symbol] .

Therefore, a transition having set of input places P_{IN} and set of output places P_{OUT} would still be linearly dependent in W′, having the same cumulative effect as \tilde{T}.

If P_{IN} ∩\tilde{P}≠Ø∧P_{OUT} ∩\tilde{P}=Ø, recall that \tilde{P} is the set of places which have the same overall effect as p′ in W′. For a linearly dependent transition \hat{t}, \tilde{T} has the same overall effect in W. In W′ if the set of input places P_{IN} and \tilde{P} are not distinct, and the set of output places P_{OUT} and \tilde{P} are distinct then, we can easily see that:

W′⋅\tilde{T}\\tilde{T}W′⋅=

[symbol]

\tilde{T}W′⋅\W′⋅\tilde{T}= [symbol] .

Hence, with respect to net W′, in order for a transition \hat{t} to be linearly dependent (i.e. have the same overall effect as \tilde{T}), it should include p′ in its input place set.

If P_{IN} ∩\tilde{P}=Ø∧P_{OUT} ∩\tilde{P} ≠Ø, recall that \tilde{P} is the set of places which have the same overall effect as p′ in W′. For a linearly dependent transition \hat{t}, \tilde{T} has the same overall effect in W. In W′ if the set of input places P_{IN} and \tilde{P} are distinct, and the set of output places P_{OUT} and \tilde{P} are not distinct then, we can easily see that:

W′⋅\tilde{T}\\tilde{T}W′⋅= [symbol] ∧

\tilde{T}W′⋅\W′⋅\tilde{T}= [symbol] .

Hence, with respect to net W′, in order for a transition \hat{t} to be linearly dependent (i.e. have the same overall effect as \tilde{T}), it should include p′ in its output place set.

If P_{IN}∩\tilde{P}≠Ø∧P_{OUT} ∩\tilde{P}≠Ø, recall that \tilde{P} is the set of places which have the same overall effect as p′ in W′. For a linearly dependent transition \hat{t}, \tilde{T} has the same overall effect in W. In W′ if the set of input places P_{IN} and \tilde{P} are not distinct, and the set of output places P_{OUT} and \tilde{P} are not distinct then, it is easy to see that P_{IN} and P_{OUT} for \hat{t} doesn't need to be updated for \hat{t} to be linearly dependent in W′. That is:

W′⋅\tilde{T}\\tilde{T}W′⋅= [symbol] ∧

\tilde{T}W′⋅\W′⋅\tilde{T}= [symbol] .

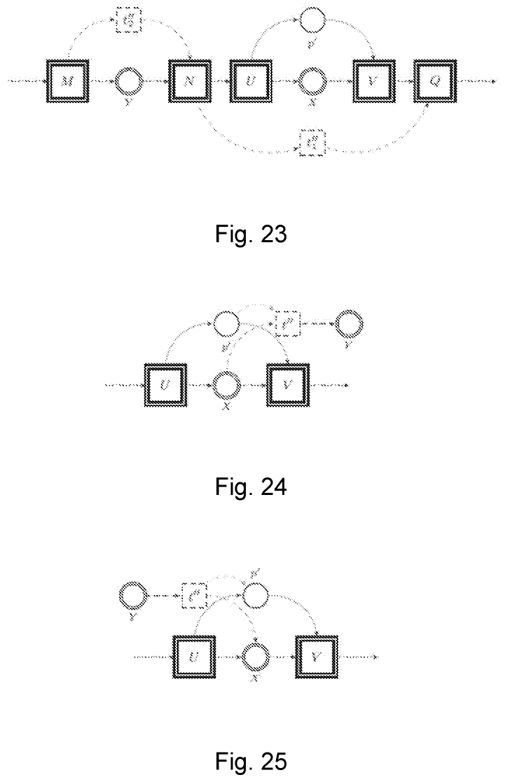

FIG. 23 shows linear dependent transitions t″₁ and t″₂ are also linearly dependent with respect to the changed net. FIG. 24 shows updating inputs of a linearly dependent transition t″, to make it linearly dependent in the new net. It should be noted that the original input to t″ could be from any S⊆X, and not necessarily from all the places in X. FIG. 15 shows updating outputs of a linearly dependent transition t″, to make it linearly dependent in the new net. It should be noted that the original input to t″ could be from any S⊆X, and not necessarily from all the places in X.

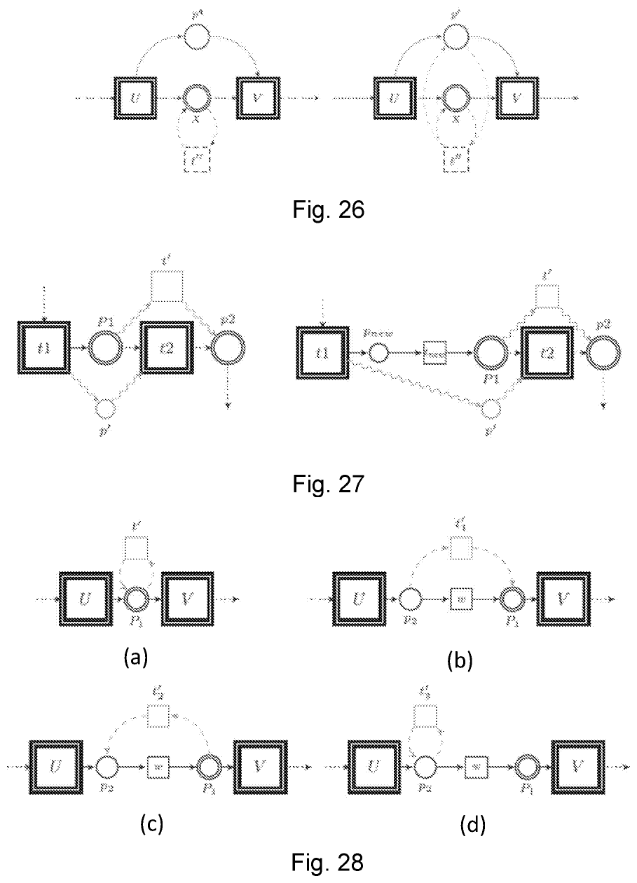

However, for such a case in Definition 32, there is an additional linearly dependent transition introduced, with set of input places P_{IN} ∪p′ and set of output places P_{OUT} ∪p′. It's easy to see that such a transition would be linearly dependent as we know that (P_{IN}, P_{OUT}) is linearly dependent, and by adding p′ in both input and output place sets, its effect is canceled out (having the value of zero in the incidence matrix). An example application usage of this rule is shown in FIG. 26.

FIG. 26 shows adding the newly added place to both the input and output of a linearly dependent transition, or not adding the place to both input and output, keeps transition t″ linearly dependent in new net. It should be noted that the original input/output to t″ could be from any S⊆X, and not necessarily from all the places in X. FIG. 26(a) shows updating neither the input nor the output of a linearly dependent transition t″ keeps it linearly dependent in the new net. FIG. 26(b) shows updating both input and output of a linearly dependent transition t″ to keep it linearly dependent in new net.

Theorem 13 and Theorem 14 state that the places and transitions extracted using Definition 32 are linearly dependent in the new net. However, we still need to prove that the updated incremental synthesis structure contains all the possible linear dependencies required in order to extract the synthesis space (SS_{P} and SS_{T}) from the new net.

Theorem 15 (The incremental synthesis space extracted using Definition 32 guarantees extraction of SS_{T}(W′) and SS_{T}(W′)).

Let W=(P, T, F, i, o) and W′=(P′, T′, F′, i′, o′) be two sound FC-WF nets, s.t. (W,W′)∈ψ′_{P}. Let ISS(N)=(TT, PP) be the incremental synthesis structures corresponding to

W. Let ISS(W′)=(TT′, PP′) be the incremental synthesis structure extracted from ISS(W) using Definition 32. Then PP′ and TT′ contain all the linearly dependent nodes required to generate synthesis space SS_{T}(W′) and SS_{T}(W′) corresponding to W′.

Proof. We first prove that the application of ψ′_{P} on a net, does not result in any new linear combinations in the net. We know that using linearly dependent rule such as ψ′_{P} does not change the rank of the incidence matrix. Since the rank is unchanged, all the previous linear dependencies still hold, and there cannot be any new linear combinations which could introduce new linear dependencies. Therefore if the initial incremental synthesis structure ISS(W) is complete to extract SS_{P}(W) and SS_{T}(W), then the new incremental synthesis structure ISS(W′) containing all the repaired elements of ISS(W) should be complete to extract SS_{P}(W′) and SS_{T}(W′) too. Definition 32 ensures that all the previous linearly dependent nodes are either updated or added as-is to the new linearly dependent node set corresponding to the new net, with the only exception being the newly added self-loop transitions (having the linearly dependent place as both input and output). From Theorem 13 and Theorem 14 we know that all the updates performed using Definition 32 result in linearly dependent nodes.

ISS updates for ‘T ISS(W′) can be extracted from ISS(W) after the application of ψ′_{T} to derive W′ from W, similar to Definition 32, as shown in Definition 33.

Definition 33 (Extracting ISS(W′) from ISS(W) after the application of ψ′_{T}).

Let W=(P, T, F, i, o) be a sound FC WF net. Let ISS(W)=(TT, PP) be the incremental synthesis structure containing all the possible linearly dependent nodes that could be added to the net W. Let W′=(P′, T′, F′, i′, o′) be a sound FC WF net synthesized from W by adding a transition t′, s.t. (W,W′)∈ψ′_{T}. Then the incremental synthesis structure ISS(W′)=(TT′, PP′) corresponding to W′ can be derived using ISS(W) as follows (note that T′\T={t′}):

[symbol]

=TT

$$- = \\ {∀}_{( {T}_{IN} , {T}_{OUT} ) ∈ PP} \\ \{ \begin{matrix}( {T}_{IN} , {T}_{OUT} ) & if \text{?} \begin{matrix}\text{?} ⋂ {T}_{IN} = ∅ ⋀ \\ \text{?} ⋂ {T}_{OUT} = ∅\end{matrix} \\ ( {T}_{IN} ⋃ \{ {t}^{′} \} , {T}_{OUT} ) & else if \text{?} \begin{matrix}\text{?} ⋂ {T}_{IN} \neq ∅ ⋀ \\ \text{?} ⋂ {T}_{OUT} = ∅\end{matrix} \\ ( {T}_{IN} , {T}_{OUT} ⋃ \{ {t}^{′} \} ) & else if \text{?} \begin{matrix}\text{?} ⋂ {T}_{IN} = ∅ ⋀ \\ \text{?} ⋂ {T}_{OUT} \neq ∅\end{matrix} \\ ( {T}_{IN} , {T}_{OUT} ) ⋃ & else if \text{?} \begin{matrix}\text{?} ⋂ {T}_{IN} \neq ∅ ⋀ \\ \text{?} ⋂ {T}_{OUT} \neq ∅\end{matrix} \\ ( {T}_{IN} ⋃ \{ {t}^{′} \} , {T}_{OUT} ⋃ \{ {t}^{′} \} ) & \end{matrix} \\ \text{?} \text{indicates text missing or illegible when filed}$$

Using Theorem 13 and Theorem 14, it can symmetrically be proven that the linear dependencies extracted after usage of ψ′_{T} are indeed linearly dependent in the new net. Furthermore, using Theorem 15 it similarly be proven that Definition 33 extracts all the possible linearly dependent nodes required to extract the synthesis space of the new net. Unlike Definition 32, Definition 33 does not introduce a new linearly dependent place with the same input and output corresponding to the newly added transition. This is for the reasons described in Corollary 4.1.

### ISS Updates for ψ′_{A}

Following the updates to the incremental synthesis structure after the usage of ψ′_{P} and ψ′_{T}, we now focus on the method used for updating the incremental synthesis structure after application of the abstraction rule ψ′_{A}. We begin with Definition 34, which defines the approach followed in order to change and update the incremental synthesis structure after the usage of ψ′_{A}.

Definition 34 (Extracting ISS(W′) from ISS(W) after the application of ψ′_{A}).

Let W=(P, T, F, i, o) be a sound FC-WF net. Let ISS(W)=(TT, PP) be the incremental synthesis structure containing all the possible linearly dependent nodes that could be added to the net W. Let W′=(P′, T′, F′, i′, o′) be a sound FC WF net synthesized from W by adding a place p′ and transition t′, s.t. (W,W′)∈ψ′ A. Let t′W′⋅=S and W′⋅p′=R. Then the incremental synthesis structure ISS(W′)=(TT′, PP′) corresponding to W′ can be derived using ISS(W) as follows (note that P′\P={p′} and T′\T={t′}):

$$+ TT ⋃ \\ ∀ \vert ( {P}_{IN} , {P}_{OUT} ) ∈ TT$$

$$\{ \begin{matrix}\{ {P}_{IN} \backslash S ⋃ \{ {p}^{′} \} , {P}_{OUT} & \vert S ⊆ {P}_{IN} \} ⋃ \\ \{ {P}_{IN} , {P}_{OUT} \backslash S ⋃ \{ {p}^{′} \} & \vert S ⊆ {P}_{OUT} \} ⋃ \\ \{ {P}_{IN} \backslash S ⋃ \{ {p}^{′} \} , {P}_{OUT} \backslash S ⋃ \{ {p}^{′} \} & \vert S ⊆ {P}_{IN} ⋀ S ⊆ {P}_{OUT} \}\end{matrix} \\ = PP ⋃ \{ ( R , {t}^{′} ) \} ⋃ \\ {∀}_{( {T}_{IN} , {T}_{OUT} ) ∈ PP} \\ \{ \begin{matrix}\{ {T}_{IN} \backslash R ⋃ \{ {t}^{′} \} , {T}_{OUT} & \vert R ⊆ {T}_{IN} \} ⋃ \\ \{ {T}_{IN} , {T}_{OUT} \backslash R ⋃ \{ {t}^{′} \} & \vert R ⊆ {T}_{OUT} \} ⋃ \\ \{ {T}_{IN} \backslash R ⋃ \{ {t}^{′} \} , {T}_{OUT} \backslash R ⋃ \{ {t}^{′} \} & \vert R ⊆ {T}_{IN} ⋀ R ⊆ {T}_{OUT} \}\end{matrix}$$

Following the Definition 34, we now provide Theorem 16, which states that all the previous linear dependencies are also valid for the new net synthesized using ψ′_{A}.

Theorem 16 (All the previous linear dependencies persist after application of ψ′ A).

Let W=(P, T, F, i, o) and W′=(P′, T′, F′, i′, o′) be two sound FC WF nets, such that (W,W′)∈ψ′_{A}. Let ISS(W)=(TT, PP) and ISS(W′)=(TT′, PP′) be the incremental synthesis structures containing all the possible linearly dependent nodes that could be added to the nets W and W′ respectively. Then ISS(W)⊂ISS(W′)

Proof. Let I(C(W)) and I(C(W′)) be the incidence matrices of nets C(W) and C(W′) respectively. We know that there exists a matrix N′ such that:

$${N}^{′} = ( \begin{matrix}I ( C ( W ) ) & B \\ 0 … 0 & - 1\end{matrix} ) where ( \begin{matrix}B \\ - 1\end{matrix} ) is the last column if I ( C ( {W}^{′} ) ) \qquad ( 1 )$$

The linear dependency set TT can be represented in terms of a matrix Tm (similar to the incidence matrix). Every pair (P_{IN}, Pₒᵤᵣ)∈TT is a column in Tₘ and the number of rows in Tₘ is the total number of places in the net W. The values in the cells of Tₘ are populated as follows: P_{IN}→{−1}, P_{OUT}→{1}, P\(P_{IN} ∪P_{OUT})→{0}, P_{IN} ∩P_{OUT}→{0}. Now, since all the columns of Tm are linearly dependent on C(W), using Equation 1 we can create a similar matrix T′ₘ s.t. each column in T′ₘ is linearly dependent in N′ as follows:

$${T}_{m}^{′} = ( \begin{matrix}{T}_{m} \\ 0 … 0\end{matrix} )$$

FIG. 27 demonstrates Theorem 16. All the previous linear dependencies persist after application of ψ′_{A}. FIG. 27(a) shows a net to which a linearly de-pendent transition t′ or a linearly dependent place p′ can be added. FIG. 27(b) shows a net derived from FIG. 27a by using ψ′_{A} and adding transition t_{new} and p_{new}. The previous linear dependencies p′ and t′ are also valid w.r.t. the new net.

∀t∈T′ₘ, t is a column in T′ₘ which is linearly dependent on N′. Therefore, for some vector x, we have:

N′·x=t

We know that there exists an invertible matrix A such that,

N′=I(C(W′))·A)

Therefore, I(C(W′))·(A·x)=t

Hence, linearly dependent transitions w.r.t. original net C(W), are also valid for the net C(W′) for a vector A·x. Therefore all linearly dependent nodes in W are also valid in W′.

It should be noted that even though all the previous linear dependencies hold after application of the abstraction rule, it merely implies that the input and output set of nodes for the existing linearly dependent transition remains the same. From Definition 31 (Paths B), we can get the sets of transitions/places in the net with respect to which the transition/place is linearly dependent. With the introduction of ψ_{A} in the net, these sets of places/transitions might have changed.

FIG. 28 is an example showing the calculation of new linearly dependent transitions after application of ψ′_{A}. FIG. 28(a) shows part of a net showing a possible linearly dependent transition t′ that can be added, such that P_{IN}=P1 and P_{OUT}=P1. FIG. 28(b) shows a net synthesized from FIG. 28a using ψ′_{A} (p2 and w are the newly added nodes). t′₁ is a linearly dependent transition with P_{1IN} and P_{1OUT} as input and output place sets resp. t′₁ is derived from t′ of FIG. 28(a) such that P_{1IN}=P_{IN} \P₁∪{p2} and P_{1OUT}=P_{OUT}. FIG. 28(c): t′₂ is a linearly dependent transition with P_{2IN} and P_{2OUT} as input and output place sets resp. t′₂ is derived from t′ of FIG. 18(a) such that P_{2IN}=P_{IN} and P2_{OUT}=P_{OUT}\P₁∪{p2}. FIG. 28(d): t′₃ is a linearly dependent transition with P_{3IN} and P_{3OUT} as input and output place sets resp. t′₃ is derived from t′ of FIG. 18(a) such that P_{3IN}=P_{IN}\P₁∪{p2} and P_{3OUT}=P_{OUT}\P₁∪{p2}.

Having established that the previous linear dependencies hold for the new net, we now prove in Theorem 17 that the updates performed in the incremental synthesis structure after the usage of ψ′_{A} are indeed linearly dependent in the new net. We follow this with a theorem, stating that Definition 34 is sufficient to extract the incremental synthesis structure required to generate the complete synthesis space of linearly dependent nodes for the new net.

Theorem 17 (Every node in ISS(W′) extracted using Definition 34 is linearly dependent in W′).

Let W=(P, T, F, i, o) and W′=(P′, T′, F′, i′, o′) be two sound FC WF nets, s.t. (W,W′)∈ψ′_{A}. Let ISS(W)=(TT, PP) be the incremental synthesis structures corresponding to W. Let a place p′ and a transition t′ be added between R and S in W to synthesize W′, such that (W, W′)∈ψ′_{A}. Let ISS(W′)=(TT′, PP′) be the incremental synthesis space up-dated extracted from ISS(W) using Definition 34. Then every node in ISS(W′) is linearly dependent in W′.

Proof. From Theorem 16, we know that all the previous set of input-output places which result in linearly dependent nodes are also valid for new net W′.

According to Definition 34, replica of every pair of sets of places (P_{IN}, P_{OUT}) in PP is created if S⊆P_{IN} or S⊆P_{OUT}. It should be noted that if S⊆P_{IN} and S⊆P_{OUT}, then we create three replicas corresponding to P_{IN}, P_{OUT}, and P_{IN},P_{OUT}. The pair of sets is recalculated for each replica by replacing S with p from the input (and/or output) place sets. Each of these replicated pair of sets is added to PP′. Consider a non-loop linearly dependent transition tᵢ containing P_{IN} and P_{OUT} as input and output place sets such that (P_{IN}, P_{OUT})∈TT and S⊆P_{OUT} (and S/⊆P_{IN}). From Theorem 16, we know that t₁ is also linearly dependent in W′. Since tᵢ is non-loop, there exists a set of transitions T∈B(W′, W′⋅ti, ti W′⋅), such that R∈T. According to Definition 34, a new linearly dependent transition \tilde{t}ᵢ, is calculated from tᵢ with input place set PIN\S∪{p} and output place set P_{OUT}. It is easy to prove that this newly added transition is indeed linearly dependent. As p′ unconditionally enables t′, and execution of t′ puts tokens in all the places of S. Now since, R∈T, and firing R would enable t′, we can say that tᵢ is linearly dependent in W′ having the same cumulative effect as T−t′. It can similarly be proven that all the other changes made using Definition 34 result in linearly dependent nodes in W′.

Theorem 18 (Incremental synthesis space extracted using Definition 34 guarantees extraction of SS_{P}(W′) and SS_{T}(W′)).

Let W=(P, T, F, i, o) and W′=(P′, T′, F′, i′, o′) be two sound FC WF nets, such that (W, W′)∈ψ′_{A}. Let ISS(W)=(TT, PP) be the incremental synthesis structure corresponding to W. Let ISS(W′)=(TT′, PP′) bet he incremental synthesis structure extracted from ISS(W) using Definition 34. Then PP′ and TT′ contains all the linearly dependent nodes required to generate the synthesis space SSP (W′) and SSTW′ corresponding to W′.

Proof. Let W′ be synthesized from W using ψ′_{A} by adding a place ‘p’ and a transition ‘t’ between transitions R and places S in W. Lets assume that there exists a linearly dependent transition x ending with the newly added place p, which cannot be derived from Definition 34 using S.

x⋅=X, s.t. p∈X,

However, t can be fired after p has a token, which would result in tokens in all the places of S. Therefore, if TT is complete, there must exist a similar linearly dependent transition x, such that:

\acute{x}⋅∈\acute{X}, s.t. ∉\acute{X},\acute{P}⊆X

Therefore, using \acute{x} in Definition 34, we can always derive the linearly dependent transition x.

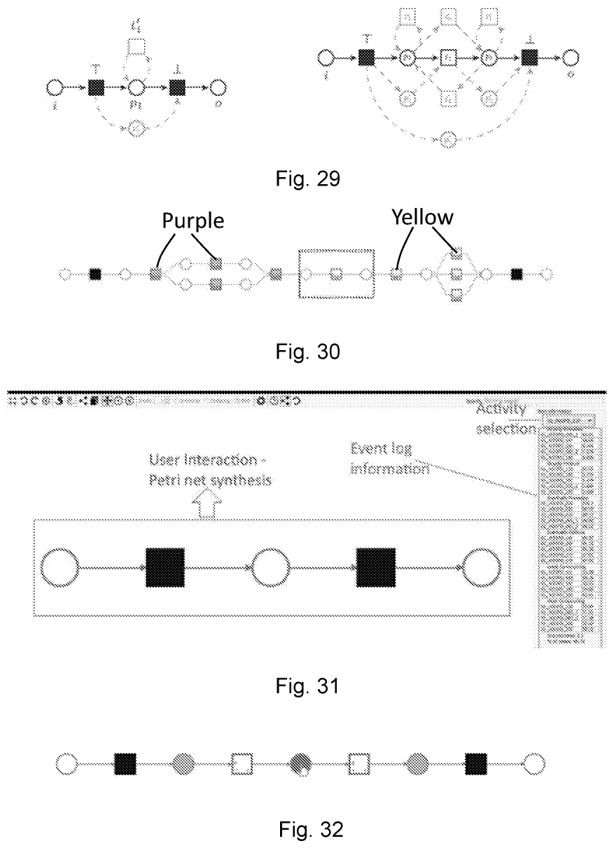

Using an incremental approach, we can successfully derive the synthesis space for every synthesized net, as shown in the sections above. However it is important to have an initial net containing the complete incremental synthesis structure. For out initial net, we have the incremental synthesis space as shown in FIG. 29. FIG. 29 shows all the linealy dependent transitions and places that could be added to our initial net. The synthesis space for linear dependencies (SS_{P}(W₀) and SS_{T}(W₀)) is the same for the initial net W₀. FIG. 29 is an example showing the use of abstraction rule on the initial net, and the possible linear dependencies that can be added to the net. FIG. 29(a) shows an initial net, showing the possible linearly dependent transition t′1 and place p′1 that can be added. FIG. 29(b) shows a net and newly created linear dependencies after using abstraction rule on FIG. 29(a).

### Event Logs Usage

In this section we discuss how the information from the event logs is presented to the user. The event logs are central for making decisions in all the automated process discovery techniques. In our case, the information from event logs is extracted and presented to the user for decision making by projecting it on the Petri net along with normalized values in a tabular form.

The synthesis rules results in the Petri net expansion one transition and/or one place at a time. The user selects the next activity from the activity list which should be added to the Petri net. Depending on the activity selected by the user, the coloring of the existing activities (transitions) in the current Petri net is updated. The colors indicate which activities (and to what extent) from the Petri net occur before and/or after the selected activity. The opacity of the colors indicate the co-occurrence of the two activities. FIG. 30 shows the usage coloring notations in our tool.

The colors and opacity of the activities are based on the eventually follows (EF) value and the co-occurs (C) value respectively. These values are calculated from the event log for all the activities in a pairwise manner using the following formulas:

Let L be an event log over Σ, i.e. L⊆Σ*. Let a, b∈Σ:

The co-occurs value C(a,b) is calculated as follows:

$${C}_{( a , b ) \vert} = \frac{\vert [ σ ∈ L \vert a ∈ σ ⋀ b ∈ σ ] \vert}{\vert [ σ ∈ L \vert a ∈ σ ] \vert}$$

a>σ b indicates a is followed by b in a trace σ and #a>σ b is the number of times b occurs after a in the trace σ
The normalized eventually follows (EF_{σ}) value for trace a and activity pair (a, b) is calculated as follows:

$${EF}_{σ} ( a , b ) = \frac{\# a {>}_{σ} b}{\# a {>}_{σ} b + \# b {>}_{σ} a} s . t . \# a {>}_{σ} b \neq 0$$

If σₗ, σ₂, σ₃, . . . , σₙ is the set of all traces in L, then the normalized eventually follows value w.r.t. the complete log L and the activity pair (a, b) is calculated as shown in FIG. 30.

FIG. 30 shows the positioning of the newly selected activity can easily be spotted by the prominent color notations. Purple colored activities occur before the selected activity and yellow colored activities occur after the selected activities. Furthermore, the activity enclosed in the dotted-box is colored white, indicating the selected activity and this activity do not occur together in any of the cases and hence must be in choice with each other.

TABLE 2
Eventually follows values for the trace t = {a, b, a, b, c, d}.

| | | EF_{σ} | EF_{σ} |
|---|---|---|---|
| Act1 | Act2 | (Act1, Act2) | (Act2, Act1) |
| a | b | 0.67 | 0.33 |
| a | c | 1 | 0 |
| a | d | 1 | 0 |
| b | a | 0.33 | 0.67 |
| . | . | . | . |
| . | . | . | . |
| . | . | . | . |

${EF}_{L} ( a , b ) = \frac{( {\sum }_{σ ∈ L} {EF}_{σ} ( a , b ) )}{\begin{matrix}\vert [ σ ∈ L \vert a ∈ σ ⋀ b ∈ σ ] \vert \\ {EF}_{L} ( a , b ) + {EF}_{L} ( b , a ) = 1\end{matrix}}$

Table 2 shows the normalized eventually follows values for a trace (σ={a, b, a, b, c, d}).

Alongside the Petri net projection indicating the position of the chosen activity, the user is also provided with aggregated activity information and six tables showing normalized numbers for better decision making. For a chosen activity, the tables show the top 5 highest normalized values for the directly following (and preceding) activities, eventually following (and preceding) activities, and most (and least) co-occurring activities. The calculations for all these tables are quite straightforward and alike. As an example, the directly follows relation w.r.t. activities a and b is calculated as follows (a>_{σ}^{d}b means a is directly followed in a trace σ):

$$Directly - Follows ( a , b ) = \frac{\vert [ σ ∈ L \vert a {>}_{σ}^{d} b ] \vert}{\vert [ σ ∈ L \vert a ∈ σ ] \vert}$$

The directly follows table for an activity a could thus be populated by calculating the pairwise directly-follows relation of a and each other activity from the event log. Besides the tabular information, the user is provided with information such as % of the traces in which the selected activity occurred and average occurrence of the selected activity in a trace.

The starting Petri net of our approach is the one shown in FIG. 29a. The user can expand the workflow net interactively in between the transitions [symbol] and ⊥. This can be easily reflected in all the traces from the event log, such that every trace starts and ends with [symbol] and ⊥ respectively. An empty trace would therefore be represented as < [symbol] ,⊥>.

### Implementation

In this section discuss the core implementation strategy used in our approach. The approach above discusses the use of synthesis space (SS(N)), which the user uses to select a synthesized model. However, in reality, navigating through all the nets from the synthesis space could be over-whelming for the user, and for large nets, unfeasible. Therefore, in our implementation, instead of directly suggesting a new net to choose from, the user interacts and is presented with the valid elements from TT and PP of ISS(N)=(TT, PP), which would result in synthesized nets of SS(N). The user interaction is described in the following steps:

1. Select an activity, to be added to the net from the activity dropbox. Alternatively, choose blank activity to add a silent transition to the net (FIG. 31).

2. Use the information from the supporting tables along with the information projected on the net as discussed in Section 5, to choose the appropriate location of the activity.

3. In order to use the abstraction rule, select the edge(s) between transition(s) and place(s), where the new place and transition should be added.

4. To add a linearly dependent place (transition), navigate over the desired input transition (place). All the possible inputs/outputs would be highlighted based on valid TT and PP and SS(N). User can click and select the desired inputs first, and the desired outputs next. FIG. 32 gives an example of this interaction.

FIG. 31 shows the screen-shot of the tool, displaying the three important sections of the tool.

FIG. 32 shows the visualization of ψ′_{T} rule in the tool. The user selects a place which would be the input of the new transition (dark grey colored place). The output could be either one of the green colored places, or the dark grey colored place (self-loop).

### Evaluation

### Conclusion and Future Research

In this paper, we presented an approach to interactively discover a process model. The use of synthesis rules to expand the Petri net guarantees soundness of the discovered model. Furthermore, we extract information from the event logs to assist user in decision making. We let the user model/discover process models by allowing the intuitive usage of the expert knowledge. Giving user complete control over the discovery approach supported by information from the event log enables critical decision making. This is true especially when all the information needed by the discovery algorithms is not present in the event log; which is often the case in many real life event logs. The automated discovery algorithms fail to cope with insufficient information in the event log, and either produce models which are incomprehensible or flower-like models. This is evident from the evaluation section, wherein our approach outperforms many of the existing process discovery techniques. Our work sits at the intersection of process modeling techniques and process discovery techniques. By combining both these worlds, we open a wide spectrum of opportunities for future research. In the future, we aim to improve the assistance provided to the user in decision making. One future direction could be to use alignments to run the event data over the user discovered model after every step and visualize the deviations. The user can thus see the impact of every change w.r.t. the event log. Another future direction could be to use any black-box machine learning algorithm such as Artificial Neural Networks to learn a model based on the data and use it in conjunction with our approach to predict the next activities that could be added in the process, thereby assisting the user in process discovery.

### ANNEX B

### Prodigy: Human-in-the-Loop Process Discovery

### Abstract

Process mining is a discipline that combines the two worlds of business process management and data mining. The central component of process mining is a graphical process model that provides an intuitive way of demonstrating the logical flow of a process. Traditionally, these process models are either modeled by a user completely relying on the domain expertise; or discovered automatically by relying entirely on the information from an event log. In an attempt to address this apparent gap between user-driven and data-driven process discovery, we present ProDiGy, an alternative approach that enables interactive process discovery by allowing the user to actively steer process discovery. ProDiGy provides the user with automatic recommendations to edit a process model, and quantify and visualize the impact of each recommendation. We evaluated ProDiGy objectively by comparing it with automated discovery approaches and subjectively by performing a user study with healthcare researchers. Our results show that ProDiGy enables inclusion of domain knowledge in process discovery, which leads to improvement of the results over the traditional process discovery techniques. Furthermore, we found that ProDiGy also increases the comprehensibility of a process model by providing the user with more control over the discovery of the process model.

### Introduction

A process is a series of actions performed in order to achieve a particular task or goal. Processes are omnipresent, and can be ad-hoc, defined explicitly or incorporated implicitly in a system. For example, a manufacturing process which deals with production of some goods, may be implicitly incorporated in the production system. On the other hand, a loan application process in a bank may be explicitly defined in a step-by-step manner in the workflow management system of the bank. A process can also be viewed at various levels of abstraction. For example, hospital visit of a patient can be represented by a very high level process common to all the patients in the hospital, by abstracting all the low level detailed activities specific to the particular patient. Alternatively, for the same patient, a different abstraction can be a process of all patients with a similar disease.

The human understanding of these processes is usually enabled by representing them as intuitive graphical models such as BPMN, EPC, Petri nets etc. Typically a domain expert who has a high level overview of the end-to-end execution of a process, models the expected process model. Since these modeling notations offer intuitive visualization of processes, they have proved to be valuable artifacts to enable communication of complex knowledge in a comprehensible way across people from dissimilar backgrounds. The process model as described by a domain expert is the best guess, of how a process acts, or should act. However, reality may not always conform to this expected behavior of the process.

The execution histories of processes, called event logs, can be easily extracted from the corresponding information systems. For example, the event logs of an administrative process can be extracted from the ERP systems, or the event logs of a careflow process in a hospital can be extracted from the EHR systems. These event logs represents a rich source of information by giving a view on a process execution in reality. Thus, these event logs can be explored to investigate any problems in the process, suggest improvements to the process etc. Statistical techniques such as statistical process control charts, are very popular particularly in the context of manufacturing processes. These techniques typically focus on analyzing the numerical data values at the activity level, and do not focus on the end-to-end process. Machine learning techniques, such as sequence mining, have also been used in order to explore common (or uncommon) sequences of process variants for analysis. However, sequence mining techniques typically miss the process context. For example, sequence mining techniques find it hard to deal with activities in a process which can occur concurrently, hence without preserving a particular sequential order.

Most of the traditional statistical or machine learning techniques either dive deep into a particular aspect of process, or miss out on the important process oriented characteristics such as choices or concurrency between activities. Process mining is a discipline which overcomes these pitfalls by taking into account the process context while analyzing the event data. Broadly, process mining can be distinguished in three categories: (i) process discovery techniques, which aim at discovering a process model using an event log; (ii) process conformance techniques, which analyze how well an event log conforms to a given process model; and (iii) process enhancement techniques, which enhance an already existing process model with information from the event log. Process conformance and enhancement techniques require a process model to work with. Hence, a process model is often the starting point for in depth process analysis.

In order to produce a process model, there are typically two options. On one end of the spectrum we have the process discovery techniques, which are automated and discover a process model directly from the event log with very limited user input. The user has very low influence over the actual process discovery, and usually it is not possible to incorporate domain knowledge during process discovery. On the other end, we have the process editor based modeling tools which are completely user driven and use no historical evidence from the event logs for process modeling. However, ideally there should be something in between that bridges the two ends. In this paper we introduce ProDiGy (Process Discovery Guided by the user), which addresses this gap in between the two approaches. ProDiGy is a tool that allows interactive discovery of a process model by including human-in-the-loop, supported by effective visualizations and data feedback. Furthermore, ProDiGy supports interactive data analytics to explore process variants.

The main contributions of this paper are:
Seamless integration of user driven and data driven approaches for process discovery, by providing recommendations to the users about possible locations of placing an activity within a process model and the impact of adding an activity at the specified location.
Auto-complete/anything in between functionality: to switch efficiently between interactive discovery and automated discovery of a process model.
Data support, feedback and analysis of the complete process as well as process variants during discovery of the process.

### Related Work

ProDiGy is an interactive process discovery system that enables human-in-the-loop process discovery. Our approach sits at the intersection of the traditional user-driven process modeling techniques and the data-driven automated process discovery techniques, and hence is closely related to these two fields. Moreover, our approach is also related to process model repair techniques which repair a process model based on the information from the event log.

### Modeling and Discovery Approaches

In this sub-section we discuss the techniques from the literature that deal either with user-driven process modeling or data-driven process discovery.

### User-Driven Modeling

Business process management as a discipline has evolved with user-driven process modeling at its core. Traditionally, a user involved in modeling of a process interviews participants across the different functions of the process, in order to get a holistic overview of the process. The knowledge gained through interviews is then used to construct a human understandable graphical process model. Many process editing tools, both open-source and commercial, have been developed to support the user in efficient editing of a process model. Most of these process editors offer users with limited or no support in decision making while modeling the process. However, with the turn of the century, there has been an added focus on recommendation based modeling support. However most of these are text-based recommendations, which are generated based on query strings or semantic similarity of activity descriptions based on ontologies, or based on historical evidence of a user's personalized preferences. That is, these techniques are not data-driven and the historical evidence from the event log is not used to generate recommendations.

### Data-Driven Process Discovery

Automatically discovering process models from event logs started gaining attention in the early 2000's with the introduction of alpha algorithm. Many discovery techniques followed. Each of these discovery algorithms tackle the process discovery problem differently, and provide different assurances. For example, some discovery techniques generate process models that guarantee perfect fitness with the event log. However, in all these approaches, the input from user is rather limited, and the decisions that lead to the generation of process models are almost entirely data-driven.

### Hybrid Approaches

The area in between manual and automated process discovery is mostly unexplored. There have been some process model repair techniques, which take as input an event log and a process model that could be hand-made; and try to minimally change the process model to reflect the reality as depicted by the event log. However the set up and goals of process model repair techniques is different compared to the human-in-the-loop process discovery. In such settings, the user has to first construct a process model without data support, which is then repaired using data. Alternatively, there have been approaches which let the user express some rules or constraints that the discovered process model must adhere to. However the user restricted in expressiveness by the language used to represent these rules. Moreover, the user has no control over the actual discovery, and the resultant process model may adhere to all the rules, but may be extremely complicated. Our approach involves users explicitly in the process of process model discovery, and hence the user can discover a process model at the preferred level of complexity.

### Preliminaries

Before diving into the details of our tool, we first introduce the preliminaries that are used throughout the paper.

### Process Models

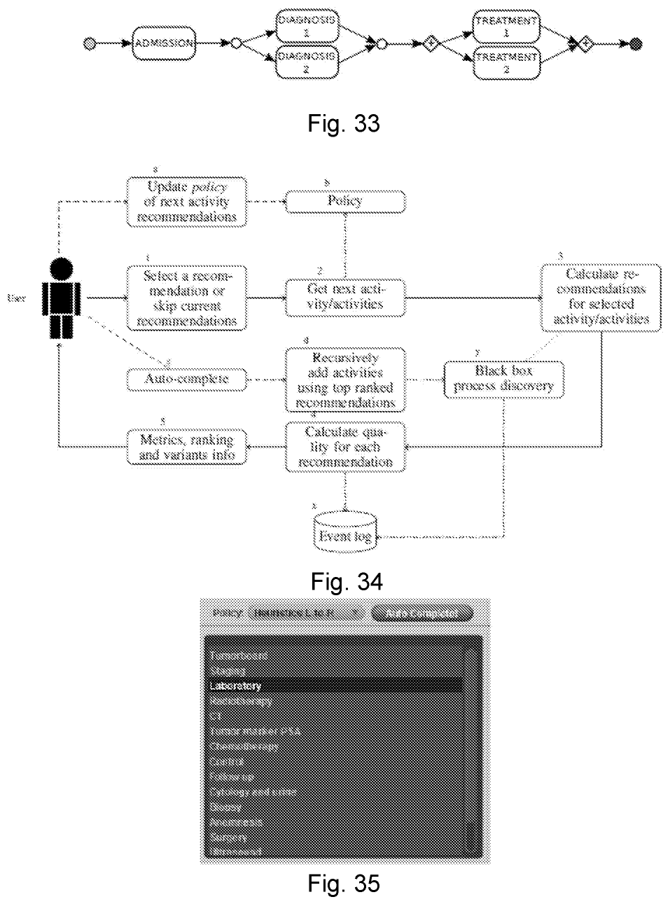

Process models are used to model the flow of steps of a process. As discussed previously, most of the process modeling approaches allow modeling of more than just sequential flow of steps. That is, they allow for rich behavioral aspects, such as choice between activities, repeatable activities—either by duplication or by introducing loops, concurrency etc. FIG. 33 shows a simple example of a process model. FIG. 33 shows a simple process model in a hospital setting. The first step in the process is Admission, followed by either one of Diagnosis 1 or Diagnosis 2. Diagnosis is followed by both Treatment 1 and Treatment 2, which can occur in any order.

### Event Logs

Event logs record the process notion of a system. Event logs record what happened i.e. an activity such as admission in the hospital, when did it happen i.e. the time when the patient was admitted, and for whom (which case), i.e. who was the patient that was admitted. Next to this, event logs may also contain additional information pertaining to cases such as the age or gender of a patient, or pertaining to activities, such as which nurse admitted the patient. Table 1 shows an example event log showing the flow of patients for process model from FIG. 33.

| Patient ID | Activity | Timestamp | Age | . . . |
|---|---|---|---|---|
| Patient 1 | Admission | 2017/02/02 14:45 | 68 | . . . |
| Patient 1 | Diagnosis 2 | 2017/02/02 15:15 | 68 | . . . |
| Patient 2 | Admission | 2017/02/02 15:18 | 45 | . . . |
| Patient 3 | Admission | 2017/02/02 15:40 | 89 | . . . |
| Patient 1 | Treatment 1 | 2017/02/02 16:41 | 68 | . . . |
| Patient 2 | Diagnosis 1 | 2017/02/02 16:45 | 45 | . . . |

### Quality Dimensions

Having introduced process models and event logs, we now discuss two standard metrics which are used to evaluate the quality of a process model based on the event log.

### Fitness

The fitness metric captures the goodness of fit of cases from an event log on a process model. For every case, the fitness value can span between 0 and 1. 1 indicates that the case can be perfectly replayed by a process model. 0 indicates that the case does not fit a process model, and the values in between 0 and 1 indicate the degree of fitness of a case and the process model. The averaged fitness values of all the cases in an event log indicate the overall fitness of an event log with a process model.

### Precision

The precision metric captures how precise a given process model is, with respect to an event log. That is, the precision metric penalizes a process model for adding extra behavior not present in the event log. Like fitness, the precision metric is scored between 0 and 1.

### Challenges of Interactive Process Discovery

This section focuses on the what, i.e. what are the key challenges addressed by ProDiGy. Human-in-the-loop process discovery aims at merging the worlds of user driven process modeling without data support; with automated process discovery which relies solely on data. In order to bridge the gap between these seemingly linked (yet fairly unexplored) disciplines, the following challenges are identified based on literature review:

C1: Recommendations for Activity Positioning in a Process Model.

Suggesting the position of an activity in a process model without overwhelming the user with too much information is one of the foremost challenges of human-in-the-loop process discovery techniques. The said activity can be placed at multiple positions within the process model. For example, suppose a new activity called Treatment 3 needs to be added to the process model from FIG. 33. Treatment 3 can happen before Treatment 1, Treatment 3 can happen after Treatment 2, Treatment 3 can happen instead of Treatment 1 and so on. Therefore it is vital to recommend to the user the locations where a particular activity can be placed in the process model.

C2: Evaluating the Recommendations.

The recommendations for changing a process model need to be evaluated based on the event log by providing relevant information about each recommendation, using the quality dimensions. Ideally the recommendations should be ranked automatically to assist the user in decision making. As an illustration, lets continue with the example from C1 of adding Treatment 3 to the model from FIG. 33. If in the event log it was observed that Treatment 3 and Treatment 1 never occur together, then recommendations suggesting anything contradictory should be penalized and ranked low.

C3: Next Possible Activity Recommendation.

Ideally, there should be a certain intuitive flow of choosing the activities during the process of process modeling. Since the process model is expanded incrementally, there should be a logical order in which the next activity (or activities) are recommended to be added in the net. For example, from a users perspective, it would be easier to get recommendations of all the related activities together (or following one another), rather than getting ad-hoc recommendations of unrelated activities.

C4: Mix and Match—Auto Discovery and Interactive Discovery.

It is important to allow the user to switch effortlessly between automated discovery and interactive discovery. The user may decide to build a process model until a certain point, and then wish to hand it over to a discovery algorithm to automatically complete the rest of the process model. Alternatively, the user may want to delegate some discovery tasks to an automated discovery algorithm, and then resume interactive process discovery.

C5: Process Variant Data Analysis.

It is common to have multiple variants of processes in a large event log. For example, a hospital event log may contain cases about different patients suffering from different diseases. In reality, some highly frequent activities may actually be specific only to certain types of diseases. Hence, a high level process model that tries to encapsulate the most frequent behavior, may cater to some diseases better than others. Thereby it is important to clearly provide such information to the user to enable informed decision making for analyzing the process variants independently, instead of just concentrating on the aggregated quality scores.

### Prodigy

This section focuses on the how, i.e. how the challenges discussed in section 4 have been addressed in ProDiGy. ProDiGy is an interactive system that automatically recommends and ranks positioning of next possible activities in a process model, while incorporating user choices at every step.

### Overview

FIG. 34 shows the high level overview of the main components of ProDiGy. The thick lines indicate the usual flow and interactions between the various components. Dashed lines indicate alternative flow and interactions between components. Dotted lines between two components from A to B indicates that component A uses component B. The usual flow and interactions between the components of FIG. 34 is as follows: the user selects one (or skips all) recommendation(s) from the list of recommendations (1), the process model is updated based on the recommendation chosen by the user and the next candidate activities are selected (2) depending on the policy chosen (b). The information from the event log (x) is used by a black box process discovery algorithm (y) to come up with different recommendations for the newly selected activity/activities (3). The quality of each recommendation is calculated (4) by using the event log (x) which is then presented to the user along with ranking and process variants information (5). At any point of time, the user can change the policy used to predict the next activity/activities (a). Similarly, the user can decide to hand over the discovery task to the automated discovery technique at any point of time (c) and (d). Auto-complete recursively adds activities to the process model by using the black box process discovery algorithm (y) by selecting the top ranked recommendation until some termination condition is met.

### Policy

Policy is a pluggable component used to decide the next activity/activities that should be added to the process model. Since process discovery in ProDiGy is incremental, the order in which the activities are added to the process model is important. There are a couple of reasons for this. Firstly, having a logical flow of adding activities incrementally may assist the user in better decision making. For example, adding all the semantically similar activities one after the other may help the user in structuring the overall process model better than adding random unrelated activities. Secondly, by following a strict order, it may become possible to discover a certain structure of process model which may otherwise become unreachable because of prior discovery decisions when adding activities randomly. As policy is a pluggable component, there could be many policies used to decide the possible next activities that are added to the process model. At any given point, the user can switch between policies by updating the policy using component a of FIG. 34. Policies essentially cater the challenge C3 from section 4. In theory, a policy can return multiple activities, and hence the recommendations presented to the user may contain process models containing some or all of those activities. However, here we present two policies which suggest a single next activity at a time. Hence throughout the upcoming sections we use the singular form of activity when referring to policies.

### Alphabetical

Alphabetical policy, as the name suggests, orders activities purely based on their alphabetical ordering. At any point, if a user decides to skip an activity, then the next activity based on the alphabetical ordering is chosen. As evident, in the case of alphabetical ordering only one activity is selected at a time. Even though this is a very simple policy, it can still be useful as the user knows which activity would follow.

### Log Heuristics

Log heuristics based policy orders activities based on the information extracted from the event log. The idea is that the user starts building the process with the most common starting activities across all the cases from the event log, and ending it with the most common ending activities across all cases in the event log. The activities in between are also ordered according to their occurrences across the cases in the event log. In this section the steps taken to order the activities using log heuristics is explained. In order to do so, we first introduce some notations.

Let L denote an event log. We know that a case is a sequence of activities. Let caseₘ be a case from the event log with the largest sequence of activities. Let σ_{casem} be the ordering sequence of activities in cₘ. Let nₘ be the total number of elements in caseₘ, that is nₘ=|σ_{casem}|.

It should be noted that the same activity can be repeated multiple times in a case, and hence can be present at multiple positions in the activity sequence of a case. As caseₘ is the case with largest sequence in the event log, no case can contain more than nₘ elements.

Let A denote the set of all the activities from the event log L. Then for any activity a∈A, let #aᵢ denote the total number of cases for which the activity at the i^{th} position is a. If a,b,c . . . ∈A, then we can compute a matrix of activity occurrences as follows:

$$F = \begin{matrix}a \\ b \\ c\end{matrix} ( \begin{matrix}1 & 2 & 3 & … & {n}_{m} \\ \# {a}_{1} & \# {a}_{2} & \# {a}_{3} & … & \# {a}_{{n}_{m}} \\ \# {b}_{1} & \# {b}_{2} & \# {b}_{3} & … & \# {b}_{{n}_{m}} \\ \# {c}_{1} & \# {c}_{2} & \# {c}_{3} & … & \# {c}_{{n}_{m}} \\ ⋮ & ⋮ & ⋮ & ⋮ & ⋮\end{matrix} )$$

Policy P of log heuristics, is a sequence of ordered activities, P=<p₁, p₂, p₃ . . . >, where p₁, p₂, p₃ . . . ∈A. In order to compute P, we introduce two functions, sort(column) and max(act).

The function sort(column) takes a number (column) as input. This corresponds to the column number of the matrix F. The function sort(column) returns an ordered list of activities sorted from highest to lowest based on the values corresponding to the column of F.

The function max(act) takes an activity act as input, and returns the maximum value from the row of the corresponding activity in the matrix F. Using the functions sort(column) and max(act), the elements of P are added incrementally as follows.

Loop from i=1 until i=nₘ
Pᵢ=sort(i)
Loop sequentially through each activity p∈Pᵢ
P=P∩{p}, iff #pᵢ=max(p)

By repeating the steps above, we end up with a policy P, which is populated by navigating the matrix F, first top down and then left to right.

### Recommendations

Recommendations component provides a list of possible edits that can be made to the current process model, to incorporate the next activity as suggested by the policy. As shown in FIG. 34, we abstract from discussing the actual process discovery approach which comes up with these recommendations as it is outside the scope of this paper. Instead we consider it a black box technique which takes as input a process model and an activity, and outputs multiple locations wherein the input activity can be added to the process model. Similar to the policy component, this black box algorithm is also a pluggable component, and any discovery algorithm which can incrementally add activities to a process model can be used. For every possible position of the activity in the process model, we then compute the fitness and precision scores using the edited process model and the event log using the alignment based conformance technique. Each recommendation has its own fitness and precision score and hence indicate the goodness of positioning an activity at various locations in the process model. The recommendations are ranked based on a ranking criteria, consisting of a weighted average of fitness and precision values. By default the precision and fitness values are weighted equally, but these weights can also be determined by the user at any given point. Recommendations and ranking component addresses the challenges C1 and C2 from section 4.

### Auto-Discovery

Auto-discovery component embeds the possibility of using traditional automated process discovery in ProDiGy. This component allows the user to pass the control to the black box discovery technique. The user provides some input to the algorithm similar to a traditional automated discovery technique, which requires some pre-configuration. The user sets the number of activities that should be added and a threshold for minimum fitness and precision values. The user can also update the weights for fitness and precision scores used for ranking the recommendations. Once these parameters are set, the auto-discovery feature iteratively adds activities to the process model, while the thresholds are met, by choosing the first ranked process model from the list of recommendations. The process models of the activities which did not satisfy the threshold criteria are skipped. After the desired number of activities are added, the user can resume the interactive process discovery. This component allows the user to switch between automated and interactive discovery and address the challenge C4 from section 4.

### Process Variants

Process variants component is used to analyze how different variants of the process behave for each recommendation. As described in the introduction of event logs, every case may have multiple features. For example a patient in a hospital, has additional attributes like age, sex, gender, type of disease etc. These features when combined with the process model can provide valuable insights. The user can compare the distribution of patients based on the process model. For example, it would be easy to investigate how a group of patients with a certain disease fit (or not fit) a given process model, compared to another group of patients with another type of disease. Since the values of fitness are pre-computed, these are re-used for providing the user with a certain feature specific distribution of cases. This component caters to the challenge C5 from section 4.

### Interface and Interaction

ProDiGy is composed of four display and interaction panels as shown in FIG. 5. In this section we describe the user interface of each panel, and the type of interactions possible that enable human-in-the-loop process discovery.

### Process Model Panel

Process model panel is panel A from FIG. 5. This is a view only panel, that is, the user does not interact directly with the process model displayed in this panel. Whenever a user chooses a recommendation, the process model in this panel is updated by placing the new activity in the process model based on the recommendation. This is the largest panel of ProDiGy. Along with the process model view, there is an option to undo a previously selected recommendation, thereby allowing the user to revert the changes made.

### Recommendations Panel

Recommendations panel contains the recommendations of possible edits to the process model based on the chosen activity. This is a tabular panel, corresponding to panel B from FIG. 5. Each row in the table corresponds to a unique way of adding an activity to the current process model. The table has three columns: rank, fitness and precision, which guide the user through the impact of each recommendation. FIG. 7 shows the primary user interaction in ProDiGy. Based on the ranking, fitness and precision scores, the user selects one row from the recommendations table. The change is animated and projected on top of the current view of process model. The layout is changed minimally, and the new nodes added to the process model are colored differently, so that the user can easily spot the part of the process model that has changed. By navigating through different rows (that is recommendations) the user can readily see the impact of each change. If a user is content with a particular recommendation, then that recommendation can be made permanent by clicking use model and select next activity button. This button finalizes a recommendation, chooses the next activity based on the policy and generates new recommendations based on the activity chosen. Alternatively the user may click skip current recommendations button to skip all of the given recommendations, choose a next activity based on the policy and generate newer recommendations based on the newly chosen activity.

### Policy Panel

Policy panel, as shown in FIG. 35, corresponds to panel C from FIG. 5. FIG. 35 shows the policy panel from ProDiGy, showing the dropdown for choosing a policy, Auto-complete button and activities list. This panel provides the user with options for choosing or updating a policy to select the next activity. Policy panel contains a policy selection box which contains all the available policies. The policy panel also contains a list box for activities which contains the activities from the event log. The user can scroll through the activities. The current activity as chosen by the policy is highlighted. Furthermore, the activities in the activity list are sorted according to the chosen policy. This provides an easy way for the user to find out the sequence of activities as suggested by the policy. The user can also interact and select any activity from the activity box. This action overrides the policy and gives user more control of the sequence in which activities should be added to the model. Whenever a new activity is chosen, either automatically by a policy, or manually by the user, the recommendations are recalculated for the newly selected activity. The policy panel also has the auto-complete button. As discussed previously, the auto-complete functionality lets the user switch between interactive discovery and automated discovery. Upon clicking the auto-complete button, the user is provided with a dialogue box to set the thresholds for minimum fitness and minimum precision values, as well as the number of activities that should be added automatically.

### Process Variants Panel

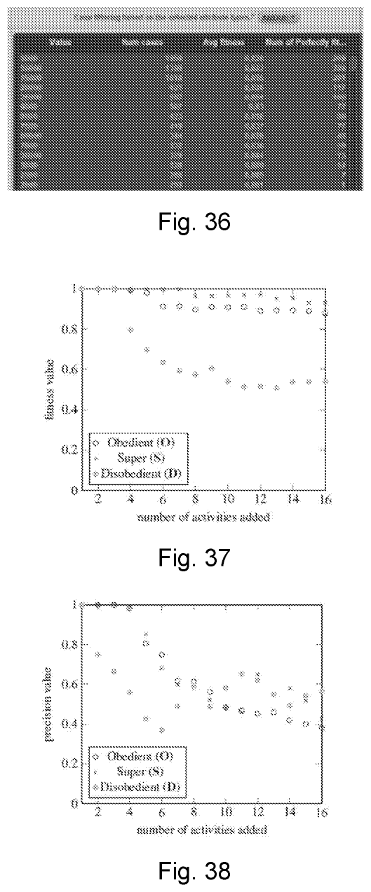

Process variants panel corresponds to panel D from FIG. 5. FIG. 36 shows the process variants tabular view populated based on the selected case level attribute AMOUNT. As shown in FIG. 36, this panel is divided into two parts. The top panel contains a feature selection list. This lets the user choose the global case feature from the list. The bottom panel shows the distribution depending on the type of feature chosen. The distribution of cases is shown in a tabular format using the process model and event log. Whenever the user chooses a certain recommendation, the information in the feature table is updated to reflect the distribution based on the changed process model.

### Design Evolution

The overall design of ProDiGy underwent a number of iterations and changes. Enabling incremental way of interactively incorporating domain knowledge was the main guiding principle behind the design of the system. After a number of revisions influenced by various methods from literature, Pro-DiGy ended up with four sub-components as discussed earlier. The idea behind designing the tool decomposed into four subcomponents is to clearly separate the steps of the workflow in the usage of the system: (i) the process model is the core component of process discovery and hence is the largest component and is placed centrally; (ii) the recommendations panel contains possible recommendations for changes in a process model. This is the first step of the workflow, wherein the user goes through the recommendations and chooses one. Since it is natural to navigate (or read through) a workflow from left-to-right, this panel is placed on the left hand side of the tool. (iii) the policy panel shows all the activities and highlights the next activity that is chosen. This next activity is chosen and highlighted after a recommendation is made permanent by the user. Hence this is typically the second step of the workflow, and hence this panel placed on the right side of the recommendations panel. (iv) the process variants panel shows the statistical information based on the impact of the current process model on the case features. The information in this panel is dependent on the activity highlighted and the recommendation chosen, and hence this panel is placed on the right side of the policy panel.

Having discussed the idea behind the overall design, and positioning of various panels, we now briefly discuss the design decisions made within some of the panels. The policy panel (panel C from FIG. 5) shows the activities as a list. The idea behind this is to let the user freely navigate through different activities, and check the natural sequence of following or preceding activities based on the chosen policy. The design of recommendations panel underwent multiple changes through several iterations. After exploring multiple design ideas, it was decided to show the changes in process model projected on the current process model. Also, by using different colors the user can easily view the impact and the change in the process model. Furthermore, it was decided to provide the recommendations in a tabular format. The tabular design was chosen to provide a holistic view showing all the recommendations, that is easy to sort and navigate.

### Implementation

ProDiGy is implemented as a plug-in in the process mining toolkit ProM. We use an alignment based approach for computing the fitness and precision values, which is also already present as a plug-in in ProM. ProDiGy can be accessed through the nightly build version of ProM, and can be downloaded from the process mining website.

### Evaluation

We conducted two types of evaluations in order to understand the usage, behavior and implications of ProDiGy. The first type of evaluation is objective and deals with analyzing a real life data set to compare the performance of various settings of ProDiGy, along with the comparison with some of the state of the art automated process discovery techniques. The second type of evaluation is subjective wherein the domain experts explore ProDiGy based on some tasks using a synthetic data set.

### Real Life Event Log

We use a real life event log to demonstrate the functionalities of ProDiGy and evaluate the outcomes of interactive process discovery. The event log used is publicly available and contains information about patients suffering from sepsis disease from a hospital. Each sepsis patient goes through a pathway of activities performed within the hospital. The information is extracted from the ERP system of the hospital and contains 15,214 activity occurrences for 16 activity classes for a total of 1050 sepsis patients.

We use ProDiGy to discover three process models. For all the three models, the weights for both fitness and precision values used during recommendations are kept at a default value of 1. Also, log heuristics based policy is chosen to predict the next activity. Using these settings, the following types of process models are discovered:

1. Obedient user (O): For each activity, the first ranked recommendation is chosen. Each activity is added only once. This corresponds to the auto-complete feature of ProDiGy.

2. Disobedient user (D): For each activity, a random recommendation is chosen from the recommendations list. Each activity is added only once. This is used to evaluate the non-conformant user who ignores the ranking criteria.

3. Super user (S): Process model is discovered by using insights about sepsis process from the literature. This can be considered a domain expert, who has a high level overview of the process and can therefore use the insights to guide the process discovery, and during the process of process discovery may choose to ignore the ranking information.

The fitness and precision values of each type of user are compared after addition of every activity to the respective process models in FIG. 37 and FIG. 38. FIG. 37 shows the change in the fitness values of the process model, after choosing a recommendation, i.e. adding an activity to the model. FIG. 38 shows the change in the precision values of the process model, after choosing a recommendation, i.e. adding an activity to the model. The fitness scores for user O and user S are fairly similar and are always above 0.9 after each iteration. The precision values for user O gradually decrease over time with each iteration. On the contrary, there is no clear pattern for the precision values of user S. This is because user S explicitly makes use of the domain knowledge, and does not rely entirely on the precision and/or fitness values. It can also be noted that both the fitness and precision scores of user S are higher than user O. User D chooses random recommendations, and this is evident in the fitness and precision scores, which follow no particular pattern and have a low value on an average. Even though the precision score of the final model (after adding activity 16) is higher for user D, the fitness score of user D is considerably lower than the other users.

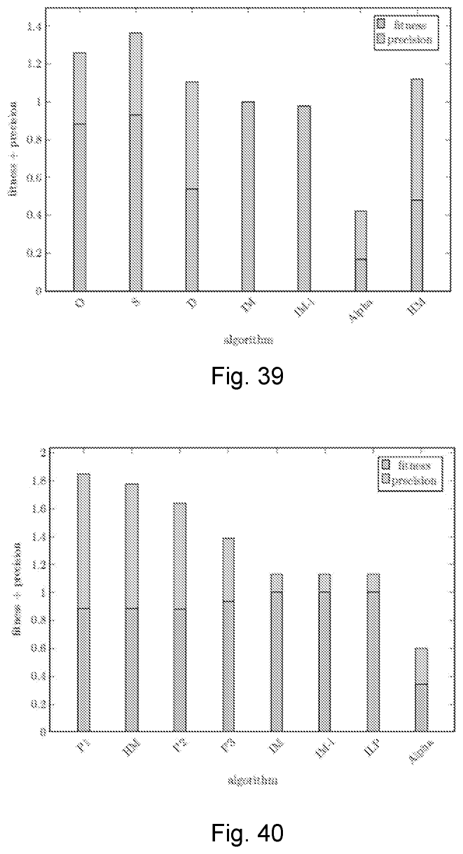

Furthermore, in order to compare the outcomes of the process models discovered using ProDiGy with the state of the art techniques, we discovered process models by using the default settings of the automated discovery techniques. Subjective measures, such as comprehensibility and generalization of the process model are inherently taken into account by the users in ProDiGy. However automated discovery techniques completely rely on the information from the event log. Hence, the automated process discovery techniques usually cannot take into account subjective measures such as simplicity of the process model. In order to keep the comparison between the approaches fair, in FIG. 39 we compare the process models entirely based on the fitness and precision values. FIG. 39 shows the fitness and precision scores of the process models discovered by obedient (O), disobedient (D), super (S) users along with the automated discovery algorithms: Inductive miner (IM), Inductive miner infrequent (IM-i), Alpha miner (Alpha), ILP Miner (ILP), Heuristics miner (HM). As evident from FIG. 39, the process models discovered using ProDiGy outperform other approaches. The outcome of obedient user (O) is comparable to the automated process discovery techniques. It should be noted that the two variants of Inductive miner (IM and IMi) find process models with almost perfect fitness. However their precision values could not be computed within a practical time frame (more than 5 hours), as these process models are too imprecise.

### User Study

Following the evaluation based on real life data, we also conducted a user study. The overall goal of the study is to understand the usability of ProDiGy, and to gain insights through user feedback. The final outputs generated by the users using ProDiGy are also evaluated and compared with the traditional automated process discovery techniques.

The user study was conducted in the context of oncology patient flow in a hospital based on simulated data. A process model was used to simulate data of 850 patients: 250 each of prostate and kidney, and 350 of bladder. Furthermore, in order to replicate the reality, noise was introduced in the data by removing 3% of random activity occurrences from random locations and adding 3% random activity occurrences at random locations in the event log. This ‘noisy’ event log was used throughout the user study. Three industry based healthcare researchers participated in this study. As a first step, all the participants were provided with a brief introduction of the tool. Next, the participants were asked to perform couple of tasks: (i) discover an end-to-end process model for all the patients with prostate cancer using the filtered (noisy) event log containing only prostate patients. (ii) discover a high level process model for all the patients using the entire (noisy) event log. The above tasks were used to guide the domain experts in exploration of ProDiGy, and their feedback was actively registered during and after their usage of the tool.

### Results

In this section, we discuss the key findings of the user evaluation.

### ProDiGy Improves the Results of Process Discovery

All the three participants were satisfied with the process models discovered by them using our tool. The comparison of fitness and precision scores for the process models discovered by the participants with some of the automatically discovered process models is shown in FIG. 40. FIG. 40 shows the fitness and precision scores of the process models discovered from the noisy event log by three participants (P1, P2, P3) along with the automated discovery algorithms: Inductive miner (IM), Inductive miner infrequent (IMi), Alpha miner (Alpha), ILP Miner (ILP), Heuristics miner (HM). The process models are evaluated on the original process model to compute the quality measures. In most of the cases, the process models discovered by the experts performed better than the automated process discovery techniques. In general, the process models discovered by the experts were deemed simpler to understand and/or more appropriate by the respective experts, compared to the process models discovered by the automated techniques. As P1 responded about one of the process models discovered automatically, “its extremely complicated and doesn't make much sense, the interactively discovered process model is easier to interpret”. All the participants agreed that ProDiGy enabled them to have more control over process discovery and suggested that the interactive process discovery enabled discovery of substantially better process models compared to the traditional automated process discovery techniques.

### Usage Patterns of ProDiGy

Even though every expert was given a standard introduction to the tool, there were differences in the way the tool was used. P1 relied almost entirely on the ranking information noting that he “trusts the data more”, and therefore he always chose either the first or second ranked recommendation from the recommendations list. Contrary to this, P2 used his domain knowledge most of the time, and relied on the recommendations when he was “unsure what to do, or where to add an activity”. P3 added the activities he was familiar with first, and then relied on the recommendations to decide the fate of unfamiliar activities. The users were satisfied with the features and the workflow of the tool, and they deemed the system easy to learn. There were suggestions made by the participants based on their specific way of discovering a process model. For example, P2 who relied more on his domain knowledge, navigated back throughout the interactive process discovery session using the undo button, and said that he “misses a redo button” to navigate forward. Similarly, P1 who relied more on the information from the event log noted that “the recommendations helped in guiding the process discovery, however sometimes the ranking difference between recommendations was hard to spot due to a very small difference in fitness and precision values”. However, in general the participants were content with the features provided. Overall, ProDiGy supported both types of users sufficiently: the one's relying on using domain knowledge as well as the one's relying on using the information from the event logs for process discovery, thereby supporting various ways of coming to a process model.

### Experts Gained Insights During Process Discovery

An unprecedented finding was that during interactively discovering the high level process model, the experts were able to gain insights in to the individual pathways of prostate, bladder and kidney cancer patients. P3 noted “the information from process variants panel assists in finding out the right overall process model, but at the same time indicates which activities are applicable for which type of patients”. Process variants information was also used by the P2 to guide the process discovery in order to “discover the process model that best fits a specific cancer type”, rather than relying only on the aggregated fitness and precision values. The strength of the traditional process discovery techniques lies in understanding the data to make certain decisions. But the decisions made during process discovery are not visible to the end user. Since our tool enables active user involvement in process discovery, it also leads to exploring patterns specific to certain features, which may otherwise remain unexplored or hidden. ProDiGy encourages users to interactively discover a process model, and can help in gathering insights during the process of process discovery, as noted by P3: “ProDiGy is surprisingly good for exploratory purposes”.

### CONCLUSION AND FUTURE WORK

The paper described a novel approach for human-in-the-loop process discovery. We first identified the key challenges of interactive process discovery and proposed solutions in order to address these challenges. The solutions proposed were designed and implemented in ProDiGy, which was tested on a real life event log and evaluated by users of healthcare domain on synthetic data. Our evaluation demonstrates that interactive process discovery can outperform the traditional automated process discovery techniques. Furthermore, the user study also demonstrated that the tool is easy to learn and the visual analytics techniques incorporated in the tool are intuitive. To our knowledge, this is the first application that involves the user during process discovery, thereby giving users more control over the comprehensibility of the final process model. In the future, we plan to extend the tool to include the suggestions made by the participants of the user study. The foremost feature to be included is a new metric to guide the user in further distinguishing the recommendations suggested, especially when the difference between the fitness and/or precision scores is not significant. Also, we plan to extend human-in-the-loop techniques to other areas of process mining, such as interactive process analytics to understand the health of a process.

1. A computer-implemented method of generating a process model, the method comprising:
displaying a generated process model to a user, the process model having been generated based on details of a first plurality of activities to be included in the process model obtained from a database and based on details of a second plurality of activities to be included in the process model received via a user input, wherein the database comprises an event log, the event log including information regarding activities that have been performed in relation to at least one previously-performed process;
obtaining from the user an activity to be included in the process model, wherein obtaining, the activity comprises determining a recommended activity based on at least an activity included in the database, suggesting the recommended activity to the user and having the user accept the recommended activity, and/or obtaining a third plurality of activities from the database, presenting the third plurality of activities to the user, and receiving a selection of an activity from the third plurality of activities;
determining one or more possible positions in the process model at which the activity to be included may be added, and presenting the possible positions to the user;
modifying the generated process model in response to receiving a selection of a possible position by adding the activity to be included at the selected position.

2. A computer-implemented method according to claim 1, further comprising:
obtaining, from a database, details of where in the process model activities are to be included.

3. (canceled)

4. (canceled)

5. A computer-implemented method according to claim 1, wherein the information comprises at least one of: information regarding the nature of an activity that has been performed; information regarding when an activity has been performed in the previously-performed process; and information regarding a subject in respect of whom an activity has been performed.

6. (canceled)

7. A computer-implemented method according to claim 1, further comprising:
ordering the third plurality of activities according to one or more metrics.

8. A computer-implemented method according to claim 7, wherein the one or more metrics are selected from a group comprising: a fitness metric, defining, for each activity in the third plurality of activities, a goodness of fit for the activity in the process model; a precision metric, defining, for each activity in the third plurality of activities, a preciseness of the process model, if the activity is included in the process model, as compared to a previously-performed process; a complexity metric, defining, for each activity in the third plurality of activities, a degree of complexity of the process model if the activity is included in the process model; and a knowledge-based metric, defining, for each activity in the third plurality of activities, at least one requirement that must be met.

9. (canceled)

10. (canceled)

11. A computer-implemented method according to claim 1, wherein the recommended activity is selected from a plurality of activities, the selection of the recommended activity being made based on at least one of: an alphabetical policy, whereby the recommended activity comprises the next activity of the plurality of activities when ordered alphabetically; and a heuristics policy, whereby the recommended activity comprises an activity which occurs most commonly in previously-performed processes.

12. (canceled)

13. A computer-implemented method according to claim 1, wherein the process model comprises a model representing a clinical care pathway.

14. A computer-implemented method according to claim 1, wherein each activity in the plurality of activities comprises one or more steps to be performed.

15. A computer program product comprising a non-transitory computer readable medium, the computer readable medium having computer readable code embodied therein, the computer readable code being configured such that, on execution by a suitable computer or processor, the computer or processor is caused to perform the method of claim 1.

16. An apparatus comprising:
a display; and
a processor configured to:
show on the display a generated process model to a user, the process model having been generated based on details of a first plurality of activities to be included in the process model obtained from a database and based on details of a second plurality of activities to be included in the process model received via a user input, wherein the database comprises an event log, the event log including information regarding activities that have been performed in relation to at least one previously-performed process;
obtain an activity to be included in the process model from the user, wherein obtaining the activity comprises determining a recommended activity based on at least an activity included in the database, suggesting the recommended activity to the user and having the user accept the recommended activity, and/or obtaining a third plurality of activities from the database, presenting the third plurality of activities to the user, and receiving a selection of an activity from the third plurality of activities;
determine one or more possible positions in the process model at which the activity to be included may be added, and presenting the possible positions to the user;
modify the generated process model in response to receiving a selection of a possible position by adding the activity to be included at the selected position.

17. A computer-implemented method according to claim 1, wherein modifying the generated process model comprises applying synthesis rules that guarantee soundness of the generated process model.

18. A computer-implemented method according to claim 11, wherein selecting the recommended activity comprises determining an ordering of activities according to occurrences, determining said ordering comprising determining an activity occurrence matrix, an entry of the activity occurrence matrix counting a number of previously-performed processes in which a certain activity occurs at a certain position, and computing the ordering therefrom.

19. A computer-implemented method according to claim 1, wherein presenting the possible positions comprises indicating which existing activities from the process model occur before and/or after the activity to be included.
